(19) United States
(12) Patent Application Publication
(10) Pub. No.: US 2026/0309689 A1
(43) Pub. Date: Oct. 8, 2026

(54) ASTROAGENTS: A MULTI-AGENT AI FOR HYPOTHESIS GENERATION FROM MASS SPECTROMETRY DATA

(71) Applicant: Georgia Tech Research Corporation, Atlanta, GA (US)

(72) Inventors: Amirali Aghazadeh, Atlanta, GA (US); Daniel Saeedi, Atlanta, GA (US)

(21) Appl. No.: 19/631,868

(22) Filed: Mar. 27, 2026

Related U.S. Application Data
(60) Provisional application No. 63/783,512, filed on Apr. 4, 2025.

(51) Int. Cl. G01N 33/24; G06N 5/043; H01J 49/26

(52) U.S. Cl. CPC G01N 33/241; G06N 5/043; H01J 49/26

(57) ABSTRACT

A method for generating hypotheses from mass spectrometry data using a multi-agent artificial intelligence system includes receiving, via a user interface, mass spectrometry data, research papers, and user instructions. The method includes analyzing, via a data analyst agent, the mass spectrometry data to identify patterns, unexpected findings, and potential contamination. The method includes delegating, via a planner agent, data segments to scientist agents based on the analysis. The method includes generating, via the scientist agents, hypotheses based on the delegated segments. The method includes consolidating, via an accumulator agent, the hypotheses by discarding duplicates. The method includes reviewing, via a literature review agent, literature relevant to each consolidated hypothesis. The method includes evaluating, via a critic agent, the consolidated hypotheses and providing feedback to the data analyst agent for iterative refinement.

### CROSS-REFERENCE TO RELATED APPLICATIONS

This application claims the benefit of U.S. Provisional Application No. 63/783,512, filed Apr. 4, 2025, entitled “SYSTEMS AND METHODS FOR HYPOTHESES GENERATION FROM MASS SPECTROMETRY DATA USING A MULTI-AGENT ARTIFICIAL INTELLIGENCE,” the entire contents of which are incorporated herein by reference.

### FIELD OF INVENTION

The present disclosure relates to systems and methods for analyzing mass spectrometry data using artificial intelligence, and more particularly to a multi-agent artificial intelligence system for generating hypotheses about molecular distributions in meteoritic and terrestrial samples from mass spectrometry data.

### BACKGROUND

Mass spectrometry is a powerful analytical technique used to identify and characterize molecular compounds in various samples. In the field of astrobiology, mass spectrometry data from sample return missions across the solar system provides valuable information about the molecular distributions of organic compounds in meteoritic and terrestrial samples. Such data can offer insights into fundamental questions about the emergence of life on Earth and the presence of organic matter in extraterrestrial environments.

The analysis of mass spectrometry data in astrobiology presents several challenges. Terrestrial contaminants can complicate the interpretation of results, as distinguishing between indigenous extraterrestrial compounds and contamination from Earth-based sources requires careful analysis. The complexity of spectral peaks in mass spectrometry data adds another layer of difficulty, as samples may contain numerous compounds with overlapping signatures. Additionally, cross-matching spectral peaks with prior studies and existing databases can be time-consuming and prone to inconsistencies.

Traditional approaches to analyzing mass spectrometry data often rely on human experts who manually interpret the results and generate hypotheses based on their knowledge and experience. However, this approach can be limited by the expertise and familiarity of individual researchers with the relevant literature. Human analysis may also be subject to cognitive biases and may not efficiently process the large volumes of high-dimensional data generated by modern mass spectrometry instruments.

Computational methods have been developed to assist with mass spectrometry data analysis, but these methods face their own challenges. The sparsity of relevant peaks relative to the dimensionality of mass spectrometry data can make pattern identification difficult. Conventional computational approaches may identify basic correlations and trends while missing more nuanced patterns that could lead to novel scientific insights.

Recent advances in artificial intelligence, particularly large language models, have demonstrated capabilities in scientific reasoning and hypothesis generation. However, individual artificial intelligence models may struggle with consistent reasoning over complex datasets, lack specialized domain expertise, and may not independently validate their outputs against scientific literature. These limitations can be particularly apparent in interdisciplinary research areas where hypotheses span multiple scientific disciplines and incorporate complex molecular evidence.

Multi-agent architectures, in which multiple artificial intelligence agents with specialized roles collaborate on tasks, have emerged as an approach to address some limitations of individual models. Such systems can combine different capabilities and knowledge domains to enhance analytical processes. However, existing multi-agent systems may lack the specialized knowledge and structured workflows suited for analyzing complex mass spectrometry data in specific scientific domains such as astrobiology.

Therefore, there exists a general desire for improved systems and methods for analyzing mass spectrometry data and generating hypotheses about molecular distributions in samples.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a method for generating hypotheses from mass spectrometry data using a multi-agent artificial intelligence system is provided. The method includes receiving, via a user interface, mass spectrometry data, one or more research papers, and instructions from a user. The method includes analyzing, via a data analyst agent, the mass spectrometry data to identify patterns, unexpected findings, and potential contamination. The method includes delegating, via a planner agent, segments of the mass spectrometry data to a plurality of scientist agents based on the analysis from the data analyst agent. The method includes generating, via the plurality of scientist agents, hypotheses based on the delegated segments of the mass spectrometry data. The method includes consolidating, via an accumulator agent, the hypotheses generated by the plurality of scientist agents by discarding duplicate hypotheses. The method includes reviewing, via a literature review agent, literature relevant to each of the consolidated hypotheses. The method includes evaluating, via a critic agent, the consolidated hypotheses based on the reviewed literature. The method includes providing feedback from the critic agent to the data analyst agent for iterative refinement of subsequent analyses.

According to other aspects of the present disclosure, the method may include one or more of the following features. Analyzing the mass spectrometry data may include identifying polycyclic aromatic hydrocarbon distribution patterns in the mass spectrometry data. The polycyclic aromatic hydrocarbon distribution patterns may include methylated polycyclic aromatic hydrocarbons and unsubstituted polycyclic aromatic hydrocarbons. The plurality of scientist agents may include three scientist agents, each scientist agent assigned to analyze a distinct research area within the mass spectrometry data. The distinct research areas may include polycyclic aromatic hydrocarbon distribution patterns, sulfur-containing and oxygen-containing compounds, and alkylated aromatic compounds. Generating the hypotheses may include outputting the hypotheses in a structured format including a hypothesis statement and data points supporting the hypothesis. The structured format may include a JSON object containing an identifier, the hypothesis statement, and the data points. Reviewing literature relevant to each of the consolidated hypotheses may include searching a scientific literature database for papers corresponding to each hypothesis and synthesizing insights from search results. Evaluating the consolidated hypotheses may include assessing alignment of each hypothesis with the mass spectrometry data, evaluating scientific rigor and theoretical basis of each hypothesis, and assessing novelty and specificity of each hypothesis. The one or more research papers may be converted to a text format and provided as background context to the data analyst agent and the plurality of scientist agents. The mass spectrometry data may include data obtained from two-dimensional gas chromatography coupled with high-resolution mass spectrometry.

According to another aspect of the present disclosure, a system for generating hypotheses from mass spectrometry data is provided. The system includes a processor. The system includes a memory coupled to the processor. The system includes a storage coupled to the processor. The storage stores instructions that, when executed by the processor, cause the processor to receive mass spectrometry data, one or more research papers, and instructions via a user interface. The instructions cause the processor to execute a data analyst agent to analyze the mass spectrometry data and identify patterns and unexpected findings. The instructions cause the processor to execute a planner agent to delegate segments of the mass spectrometry data to a plurality of scientist agents. The instructions cause the processor to execute the plurality of scientist agents to generate hypotheses based on assigned segments of the mass spectrometry data. The instructions cause the processor to execute an accumulator agent to consolidate the hypotheses by removing duplicate hypotheses. The instructions cause the processor to execute a literature review agent to search for and summarize literature relevant to each consolidated hypothesis. The instructions cause the processor to execute a critic agent to evaluate the consolidated hypotheses and provide feedback to the data analyst agent for iterative refinement.

According to other aspects of the present disclosure, the system may include one or more of the following features. The instructions may further cause the processor to convert the one or more research papers to a text format and provide the text format as background context to the data analyst agent and the plurality of scientist agents. The plurality of scientist agents may include three scientist agents, each scientist agent configured to analyze a distinct research area within the mass spectrometry data. The distinct research areas may include polycyclic aromatic hydrocarbon distribution patterns, sulfur-containing and oxygen-containing compounds, and alkylated aromatic compounds. The instructions may cause the processor to execute the critic agent to evaluate the consolidated hypotheses by assessing alignment of each hypothesis with the mass spectrometry data, evaluating scientific rigor and theoretical basis of each hypothesis, assessing novelty and specificity of each hypothesis, and generating structured feedback for iterative refinement. The instructions may cause the processor to execute the literature review agent to search a scientific literature database for papers corresponding to each consolidated hypothesis and synthesize insights from search results.

According to another aspect of the present disclosure, a non-transitory computer-readable medium storing instructions is provided. The instructions, when executed by a processor, cause the processor to perform operations including receiving mass spectrometry data and research papers. The operations include analyzing, via a data analyst agent, the mass spectrometry data to identify patterns. The operations include delegating, via a planner agent, data segments to a plurality of scientist agents. The operations include generating, via the plurality of scientist agents, hypotheses based on the delegated data segments. The operations include consolidating, via an accumulator agent, the generated hypotheses. The operations include reviewing, via a literature review agent, literature for each consolidated hypothesis. The operations include evaluating, via a critic agent, the consolidated hypotheses. The operations include iteratively refining the analysis based on feedback from the critic agent.

According to other aspects of the present disclosure, the non-transitory computer-readable medium may include one or more of the following features. The operations may further include converting the research papers to a text format and providing the text format as background context to the data analyst agent and the plurality of scientist agents. The plurality of scientist agents may include three scientist agents, each scientist agent configured to analyze a distinct research area within the mass spectrometry data.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a block diagram of a method for generating hypotheses using a multi-agent artificial intelligence system, according to aspects of the present disclosure.

FIG. 1A illustrates the user interface of the method for generating hypotheses, according to aspects of the present disclosure.

FIG. 1B illustrates the data analyst agent of the method for generating hypotheses, according to an embodiment.

FIG. 1C illustrates the planner agent of the method for generating hypotheses, according to aspects of the present disclosure.

FIG. 1D illustrates the scientist agents of the method for generating hypotheses, according to an embodiment.

FIG. 1E illustrates the accumulator agent of the method for generating hypotheses, according to aspects of the present disclosure.

FIG. 1F illustrates the literature review agent of the method for generating hypotheses, according to an embodiment.

FIG. 1G illustrates the critic agent of the method for generating hypotheses, according to aspects of the present disclosure.

FIG. 2A depicts a data analyst agent prompt interface for the multi-agent artificial intelligence system, according to an embodiment.

FIG. 2B depicts a data analyst agent output for the multi-agent artificial intelligence system, according to aspects of the present disclosure.

FIG. 3A depicts a planner agent prompt interface for the multi-agent artificial intelligence system, according to aspects of the present disclosure.

FIG. 3B depicts a planner agent output for the multi-agent artificial intelligence system of FIG. 3A, according to an embodiment.

FIG. 4A depicts a scientist agent prompt interface for the multi-agent artificial intelligence system, according to aspects of the present disclosure.

FIG. 4B depicts a scientist agent output for the multi-agent artificial intelligence system of FIG. 4A, according to an embodiment.

FIG. 5A depicts a literature review agent prompt interface for the multi-agent artificial intelligence system, according to aspects of the present disclosure.

FIG. 5B depicts a literature review agent output for the multi-agent artificial intelligence system of FIG. 5A, according to an embodiment.

FIG. 6A depicts a critic agent prompt interface configured for a data analyst agent, according to aspects of the present disclosure.

FIG. 6B depicts a critic agent prompt interface configured for a literature review agent, according to an embodiment.

FIG. 6C depicts a critic agent prompt interface configured for a scientist agent, according to aspects of the present disclosure.

FIG. 6D depicts a critic agent prompt interface configured as an accumulator agent, according to an embodiment.

FIG. 6E depicts a critic agent prompt interface configured for a planner agent, according to aspects of the present disclosure.

FIG. 6F depicts a critic agent prompt interface for hypothesis evaluation, according to an embodiment.

FIG. 7 illustrates a flowchart of a method for generating hypotheses from mass spectrometry data, according to aspects of the present disclosure.

FIG. 8 illustrates a flowchart of a method for analyzing mass spectrometry data using a data analyst agent, according to an embodiment.

FIG. 9 illustrates a flowchart of a method for delegating data analysis using a planner agent, according to aspects of the present disclosure.

FIG. 10 illustrates a flowchart of a method for generating hypotheses using hypothesis generation agents, according to an embodiment.

FIG. 11 illustrates a flowchart of a method for consolidating hypotheses using an accumulator agent, according to aspects of the present disclosure.

FIG. 12 illustrates a flowchart of a method for reviewing literature using a literature review agent, according to an embodiment.

FIG. 13 illustrates a flowchart of a method for evaluating hypotheses using a critic agent, according to aspects of the present disclosure.

FIG. 14 illustrates a block diagram of a computing system for implementing the multi-agent artificial intelligence system, according to an embodiment.

FIG. 15 illustrates a network diagram for the multi-agent artificial intelligence system, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to a multi-agent artificial intelligence system configured to generate hypotheses from mass spectrometry data. The system may be used to analyze molecular distributions in samples, including meteoritic samples and terrestrial samples. In some cases, the system facilitates the interpretation of complex, high-dimensional mass spectrometry datasets by employing multiple specialized agents that collaborate to identify patterns, generate hypotheses, and evaluate those hypotheses against existing scientific literature.

Mass spectrometry data from sample return missions and other sources may contain traces of organic compounds that provide information about the origins of life on Earth. Analyzing such data presents challenges due to the presence of terrestrial contaminants, the complexity of spectral peaks, and the lack of systematic approaches for hypothesis generation. The multi-agent artificial intelligence system described herein addresses these challenges by providing a structured workflow for data analysis and hypothesis generation.

The multi-agent artificial intelligence system may comprise a plurality of agents, each agent configured to perform a specialized function within the hypothesis generation workflow. In some cases, the agents include a data analyst agent, a planner agent, one or more scientist agents, an accumulator agent, a literature review agent, and a critic agent. Each agent may operate on data received from other agents in the system, and the agents may collaborate to produce refined hypotheses that are supported by both the mass spectrometry data and existing scientific literature.

The system may receive input from a user, including mass spectrometry data, research papers, and instructions. The mass spectrometry data may include compound identifiers, mass-to-charge ratios, retention times, compound names, and sample information. The research papers may provide background context for the analysis and hypothesis generation. In some cases, the research papers are converted to a text format and provided to the agents as background knowledge.

The multi-agent artificial intelligence system may employ an iterative refinement process in which feedback from the critic agent is provided to the data analyst agent to enhance subsequent analyses and hypothesis generation. This iterative approach may improve the quality of generated hypotheses over multiple iterations by incorporating critiques and suggestions for improvement.

In some cases, the system is configured to analyze mass spectrometry data obtained from two-dimensional gas chromatography coupled with high-resolution mass spectrometry. The system may be compatible with mass spectrometry data from various instruments and may be applied to datasets beyond those specifically described herein.

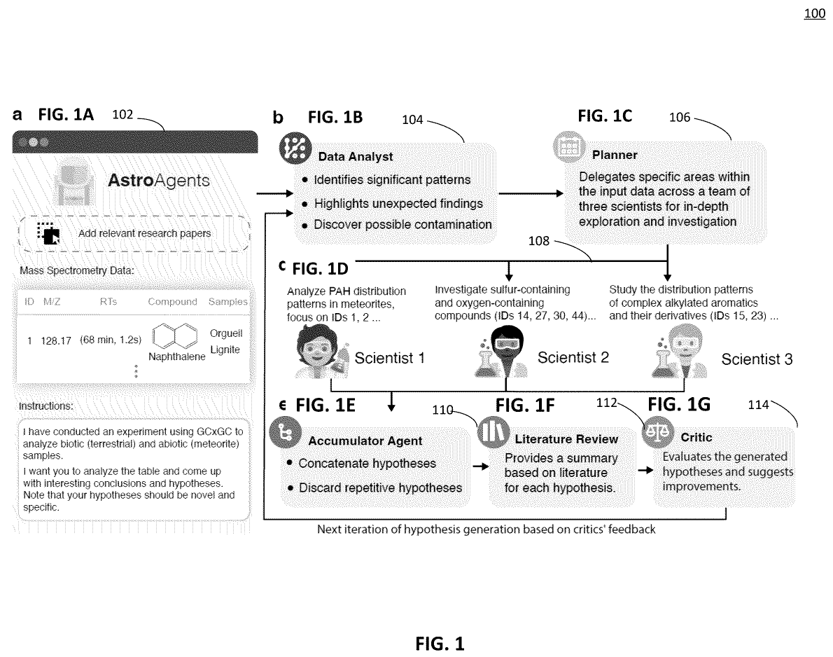

Referring to FIG. 1, a method 100 for generating hypotheses from mass spectrometry data using a multi-agent artificial intelligence system is illustrated. The method 100 includes a user interface 102, a data analyst agent 104, a planner agent 106, scientist agents 108, an accumulator agent 110, a literature review agent 112, and a critic agent 114. The method 100 provides a structured workflow for receiving mass spectrometry data, analyzing the mass spectrometry data through multiple specialized agents, and generating hypotheses through an iterative refinement process.

The user interface 102 may receive mass spectrometry data, one or more research papers, and instructions from a user. As shown in FIG. 1A, the user interface 102 displays an application that allows users to add relevant research papers and view mass spectrometry data in a tabular format. The mass spectrometry data may include compound identifiers, mass-to-charge ratios, retention times, compound names, and sample information. The one or more research papers may be converted to a text format and provided as background context to the data analyst agent 104 and the scientist agents 108.

With continued reference to FIG. 1, the data analyst agent 104 receives input from the user interface 102 and performs analysis on the mass spectrometry data. As shown in FIG. 1B, the data analyst agent 104 may identify patterns in the mass spectrometry data, detect unexpected findings, and discover possible contamination within the samples. The data analyst agent 104 refines its findings based on feedback from the critic agent 114 in an iterative process, enabling enhanced analyses over multiple iterations.

As shown in FIG. 1C, the planner agent 106 receives output from the data analyst agent 104 and delegates segments of the mass spectrometry data to the scientist agents 108 based on the analysis from the data analyst agent 104. The planner agent 106 may assign distinct research focuses to each of the scientist agents 108, allowing for in-depth exploration of different aspects of the mass spectrometry data.

As shown in FIG. 1D, the scientist agents 108 may include a plurality of agents, each agent assigned to analyze different aspects of the mass spectrometry data as directed by the planner agent 106. The scientist agents 108 generate hypotheses based on the delegated segments of the mass spectrometry data. Each of the scientist agents 108 may focus on a specific research area, such as polycyclic aromatic hydrocarbon distribution patterns, sulfur-containing and oxygen-containing compounds, or alkylated aromatic compounds.

As shown in FIG. 1E, the accumulator agent 110 receives hypotheses generated by the scientist agents 108 and consolidates the hypotheses by discarding duplicate hypotheses. The accumulator agent 110 may identify and remove substantially similar hypotheses to produce a consolidated set of unique hypotheses for further evaluation.

As shown in FIG. 1F, the literature review agent 112 receives the consolidated hypotheses from the accumulator agent 110 and reviews literature relevant to each of the consolidated hypotheses. The literature review agent 112 may search a scientific literature database for papers corresponding to each hypothesis and synthesize findings from the search results. The literature review agent 112 incorporates vast corpora of scientific literature, generating insights beyond the capacity of human analysis.

As shown in FIG. 1G, the critic agent 114 receives output from the literature review agent 112 and evaluates the consolidated hypotheses based on the reviewed literature. The critic agent 114 may assess alignment of each hypothesis with the mass spectrometry data, evaluate scientific rigor and theoretical basis, and assess novelty and specificity of the claims. The critic agent 114 provides feedback to the data analyst agent 104 for iterative refinement of subsequent analyses.

The method 100 jointly operates on both mass spectrometry data and text data for comprehensive analysis. The system may be compatible with mass spectrometry instruments of various types, ensuring broad applicability across platforms. In some cases, the method 100 enables hypothesis generation without requiring prior domain expertise in mass spectrometry, making advanced analysis accessible to users who may not have specialized training in mass spectrometry data interpretation.

A system for generating hypotheses from mass spectrometry data may implement the method 100 using a processor, a memory coupled to the processor, and a storage coupled to the processor. The storage may store instructions that, when executed by the processor, cause the processor to receive mass spectrometry data, one or more research papers, and instructions via the user interface 102, and to execute the data analyst agent 104, the planner agent 106, the scientist agents 108, the accumulator agent 110, the literature review agent 112, and the critic agent 114. A non-transitory computer-readable medium may store instructions that, when executed by a processor, cause the processor to perform operations comprising receiving mass spectrometry data and research papers, and executing the agents of the method 100 to generate hypotheses.

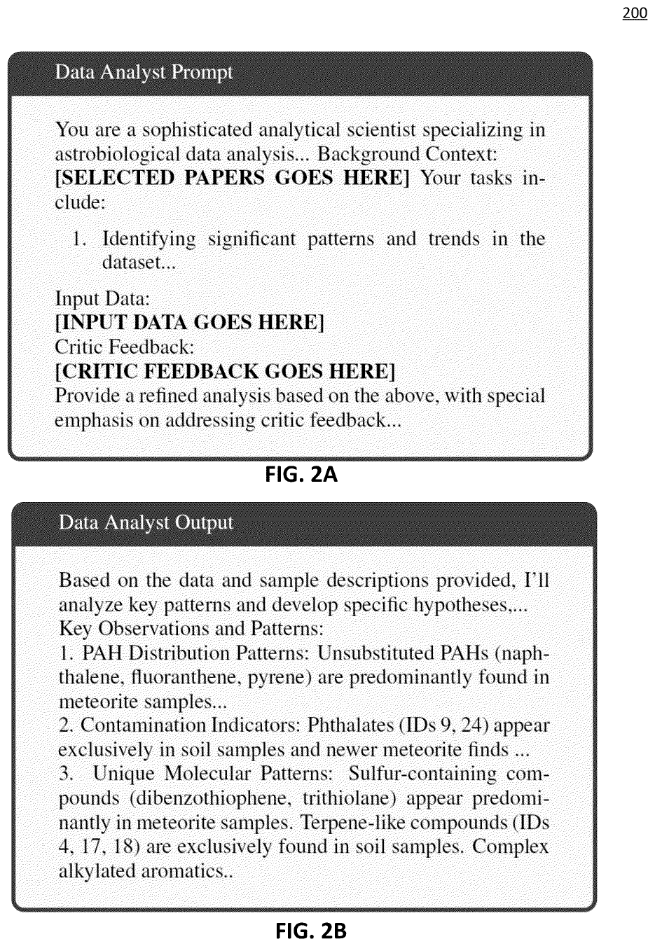

Referring to FIG. 2A, a data analyst agent prompt interface 200 is illustrated. The data analyst agent prompt interface 200 provides a structured prompt template for the data analyst agent 104 to analyze mass spectrometry data. The data analyst agent prompt interface 200 includes a data analyst prompt section, an input data section, a critic feedback section, and a data analyst output section.

The data analyst agent 104 employs the structured prompt template of the data analyst agent prompt interface 200 to access mass spectrometry data, astrobiological context extracted from academic papers, and user instructions. The structured prompt template may instruct the data analyst agent 104 to function as a sophisticated analytical scientist specializing in astrobiological data analysis. In some cases, the data analyst agent prompt interface 200 specifies that background context from selected papers is provided to the data analyst agent 104.

With continued reference to FIG. 2A, the data analyst agent 104 analyzes the mass spectrometry data to identify patterns, unexpected findings, and potential contamination. The data analyst agent 104 identifies patterns in the mass spectrometry data by examining compound distributions across different samples. In some cases, analyzing the mass spectrometry data comprises identifying polycyclic aromatic hydrocarbon distribution patterns in the mass spectrometry data. The polycyclic aromatic hydrocarbon distribution patterns may include methylated polycyclic aromatic hydrocarbons and unsubstituted polycyclic aromatic hydrocarbons.

The data analyst agent 104 identifies polycyclic aromatic hydrocarbon distributions and alkylation patterns within the mass spectrometry data. As shown in the data analyst output section of the data analyst agent prompt interface 200, the data analyst agent 104 may identify that unsubstituted polycyclic aromatic hydrocarbons such as naphthalene, fluoranthene, and pyrene are predominantly found in meteorite samples. The data analyst agent 104 may further identify alkylation patterns by detecting methylated polycyclic aromatic hydrocarbons in the mass spectrometry data.

The data analyst agent 104 identifies possible environmental contamination in the samples by considering terrestrial versus extraterrestrial signatures. As further shown in FIG. 2A, the data analyst agent prompt interface 200 displays contamination indicators in the data analyst output section. The data analyst agent 104 may detect that phthalates appear in soil samples and newer meteorite finds, indicating potential terrestrial contamination. The data analyst agent 104 distinguishes between compounds that are likely of extraterrestrial origin and compounds that may represent terrestrial contamination.

The data analyst agent 104 highlights unexpected or unusual findings in the mass spectrometry data, particularly regarding temperature indicators. The data analyst agent 104 may identify unique molecular patterns that suggest formation conditions or thermal histories of the samples. In some cases, the data analyst agent 104 detects sulfur-containing compounds such as dibenzothiophene and trithiolane that appear predominantly in meteorite samples, which may indicate specific formation environments.

The data analyst agent 104 compares data subsets within the mass spectrometry data, especially between different meteorite classes. The data analyst agent 104 may compare compound distributions between CM2 chondrites, CII meteorites, and other meteorite classifications to identify class-specific patterns. The data analyst agent prompt interface 200 facilitates this comparison by providing the data analyst agent 104 with access to sample information that identifies the meteorite class of each sample.

The data analyst agent prompt interface 200 includes a critic feedback section where feedback from the critic agent 114 is inserted. The data analyst agent 104 refines its analysis based on the critic feedback, with instructions to provide a refined analysis with emphasis on addressing the critic feedback. The data analyst agent 104 may pay attention to rewarded aspects and avoid patterns similar to criticized aspects in subsequent iterations.

Referring to FIG. 2B, a data analyst agent output for the multi-agent artificial intelligence system is illustrated. The data analyst agent output displays key observations and patterns identified by the data analyst agent 104 from the mass spectrometry data. The data analyst agent output provides the foundation for the planner agent 106 to delegate research areas to the scientist agents 108.

The data analyst agent output includes a first observation regarding polycyclic aromatic hydrocarbon distribution patterns. The data analyst agent 104 identifies that unsubstituted polycyclic aromatic hydrocarbons such as naphthalene, fluoranthene, and pyrene are predominantly found in meteorite samples. The polycyclic aromatic hydrocarbon distribution patterns provide information about the molecular signatures that distinguish meteoritic samples from terrestrial samples.

The data analyst agent output includes a second observation regarding contamination indicators. The data analyst agent 104 identifies that phthalates with compound identifiers 9 and 24 appear exclusively in soil samples and newer meteorite finds. The contamination indicators enable the data analyst agent 104 to distinguish between compounds that are likely of extraterrestrial origin and compounds that may represent terrestrial contamination.

The data analyst agent output includes a third observation regarding unique molecular patterns. The data analyst agent 104 identifies that sulfur-containing compounds such as dibenzothiophene and trithiolane appear predominantly in meteorite samples. The data analyst agent 104 further identifies that terpene-like compounds with compound identifiers 4, 17, and 18 are exclusively found in soil samples. The data analyst agent output also identifies complex alkylated aromatics within the mass spectrometry data.

The data analyst agent output shown in FIG. 2B enables the planner agent 106 to identify distinct research areas within the mass spectrometry data. The key observations and patterns identified by the data analyst agent 104 inform the delegation of data segments to the scientist agents 108 for hypothesis generation.

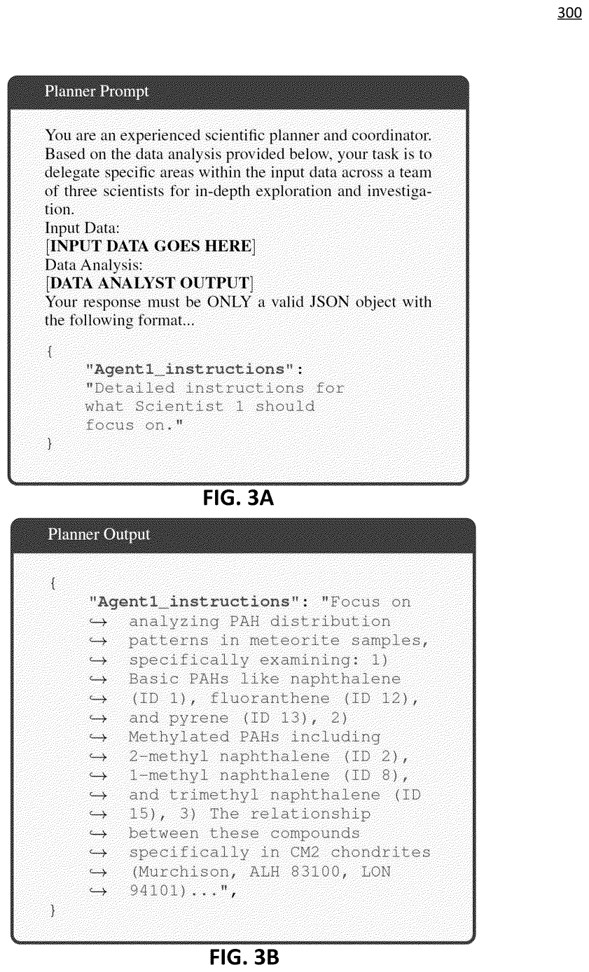

Referring to FIG. 3A and FIG. 3B, a planner agent prompt interface 300 is illustrated. The planner agent prompt interface 300 provides a structured prompt template and corresponding output format for the planner agent 106 to delegate segments of the mass spectrometry data to a plurality of scientist agents based on the analysis from the data analyst agent 104. The planner agent prompt interface 300 includes a planner prompt section and a planner output section.

The planner agent 106 receives output from the data analyst agent 104 and delegates segments of the mass spectrometry data to the scientist agents 108. As shown in FIG. 3A, the planner agent prompt interface 300 instructs the planner agent 106 to function as an experienced scientific planner and coordinator. The planner agent 106 receives input data and data analyst output, and the planner agent 106 is tasked with delegating specific areas within the input data across a team of scientist agents for in-depth exploration and investigation.

With continued reference to FIG. 3A, the planner agent prompt interface 300 includes placeholders for input data and data analyst output. The planner agent 106 analyzes the data analyst output to identify distinct research areas within the mass spectrometry data. The planner agent 106 then generates instructions for each of the scientist agents 108 in JSON format. The planner agent prompt interface 300 specifies that the response from the planner agent 106 is a valid JSON object containing instruction fields for each scientist agent.

The plurality of scientist agents 108 may comprise three scientist agents, each scientist agent assigned to analyze a distinct research area within the mass spectrometry data. As shown in FIG. 3B, the planner agent prompt interface 300 displays a sample JSON response containing Agent1_instructions, Agent2_instructions, and Agent3_instructions fields. Each instruction field provides detailed instructions for what the respective scientist agent should focus on during hypothesis generation.

The distinct research areas may include polycyclic aromatic hydrocarbon distribution patterns, sulfur-containing and oxygen-containing compounds, and alkylated aromatic compounds. As further shown in FIG. 3B, the planner output section of the planner agent prompt interface 300 displays Agent1_instructions that direct a first scientist agent to focus on analyzing polycyclic aromatic hydrocarbon distribution patterns in meteorite samples. The Agent1_instructions may specify examination of basic polycyclic aromatic hydrocarbons such as naphthalene, fluoranthene, and pyrene, as well as methylated polycyclic aromatic hydrocarbons including 2-methyl naphthalene, 1-methyl naphthalene, and trimethyl naphthalene.

The planner agent 106 assigns a second scientist agent to investigate sulfur-containing and oxygen-containing compounds within the mass spectrometry data. The planner agent 106 assigns a third scientist agent to study the distribution patterns of alkylated aromatic compounds and their derivatives. Each scientist agent receives compound identifiers in the task splits from the planner agent 106, enabling focused analysis of specific compounds within the mass spectrometry data.

The planner agent prompt interface 300 includes guidelines for the planner agent 106 to follow when generating instructions. The planner agent 106 focuses on data analysis and divides work among the three scientist agents. The planner agent 106 includes compound identifiers in the task splits to direct each scientist agent to specific compounds for analysis. The planner agent 106 excludes environmental contamination from task splits, as contamination analysis is performed by the data analyst agent 104.

The JSON format instructions generated by the planner agent 106 enable each scientist agent to receive structured directives for hypothesis generation. The planner agent prompt interface 300 specifies that the response from the planner agent 106 contains no additional text before or after the JSON object, ensuring that the scientist agents 108 receive properly formatted instructions for processing.

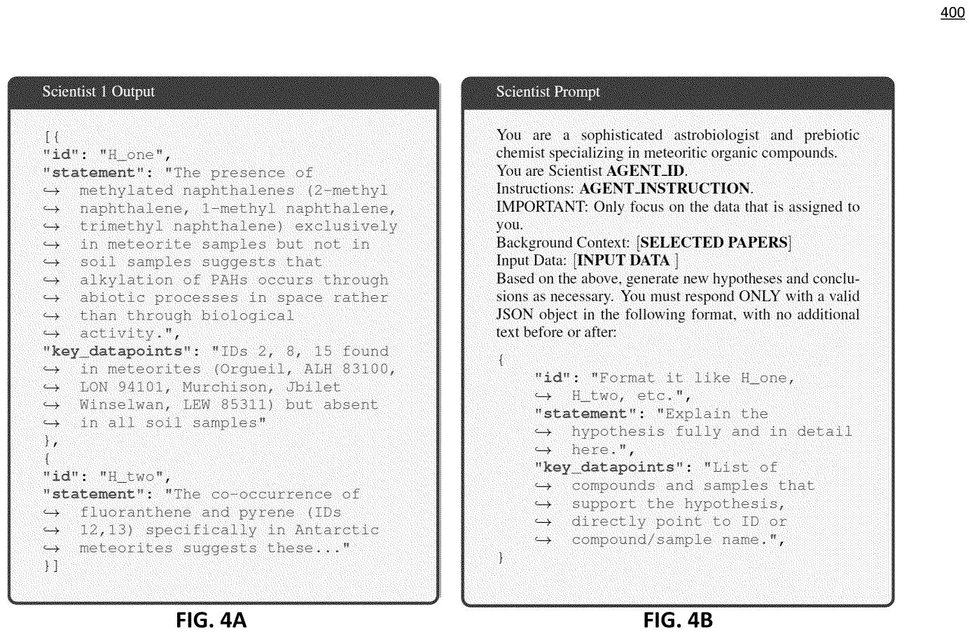

Referring to FIG. 4A and FIG. 4B, a scientist agent prompt interface and a corresponding scientist agent output are illustrated for the multi-agent artificial intelligence system for hypothesis generation from mass spectrometry data. FIG. 4A depicts a scientist prompt that establishes the role of each scientist agent as a sophisticated astrobiologist and prebiotic chemist specializing in meteoritic organic compounds. FIG. 4B depicts a scientist output containing hypotheses generated by the scientist agent in a structured format.

As described previously, the plurality of scientist agents generate hypotheses based on the delegated segments of the mass spectrometry data. Each scientist agent receives instructions and an assigned research area from the planner agent. The scientist prompt shown in FIG. 4A assigns each scientist agent a specific identifier and provides instructions to focus on assigned data. The scientist prompt specifies that background context from selected papers and input data are provided to each scientist agent.

With continued reference to FIG. 4A, the scientist prompt instructs each scientist agent to generate hypotheses and conclusions from the input data. The scientist prompt specifies that each scientist agent is to be original and novel while considering established formation mechanisms. Each scientist agent makes conclusions based on the input data and the instructions received from the planner agent. The scientist prompt further specifies that each scientist agent does not include environmental contamination in hypotheses, as contamination analysis is performed by the data analyst agent.

Generating the hypotheses comprises outputting the hypotheses in a structured format including a hypothesis statement and key data points supporting the hypothesis. As shown in FIG. 4A, the scientist prompt requires each scientist agent to respond with a valid JSON object in a specified format. The structured format comprises a JSON object containing an identifier, the hypothesis statement, and the key data points. The identifier may be formatted using a naming convention such as H\_one, H\_two, or similar designations to distinguish between different hypotheses.

As further shown in FIG. 4B, the scientist agent output contains hypothesis entries in the structured JSON format. The scientist agents generate hypotheses in a structured JSON format containing an identifier, statement, and key datapoints. Each hypothesis entry includes an identifier field that provides a unique designation for the hypothesis, a statement field that explains the hypothesis fully and in detail, and a key\_datapoints field that lists compounds and samples that support the hypothesis with direct references to compound identifiers or sample names.

The scientist agent output shown in FIG. 4B includes a first hypothesis with identifier H\_one. The first hypothesis states that the presence of methylated naphthalenes including 2-methyl naphthalene, 1-methyl naphthalene, and trimethyl naphthalene in meteorite samples but not in soil samples suggests that alkylation of polycyclic aromatic hydrocarbons occurs through abiotic processes in space rather than through biological activity. The key datapoints for the first hypothesis reference identifiers 2, 8, and 15 found in meteorites including Orgueil, ALH 83100, LON 94101, Murchison, Jbilet Winselwan, and LEW 85311 but absent in all soil samples.

With continued reference to FIG. 4B, the scientist agent output includes a second hypothesis with identifier H\_two. The second hypothesis describes the co-occurrence of fluoranthene and pyrene with identifiers 12 and 13 in Antarctic meteorites. Each hypothesis generated by the scientist agents includes supporting evidence in the form of key data points that substantiate the hypothesis, enabling evaluation of the hypothesis against the mass spectrometry data.

The plurality of scientist agents may comprise three scientist agents, each scientist agent configured to analyze a distinct research area within the mass spectrometry data. As described previously, the distinct research areas may include polycyclic aromatic hydrocarbon distribution patterns, sulfur-containing and oxygen-containing compounds, and alkylated aromatic compounds. Each scientist agent operates within its assigned domain to generate hypotheses that are specific to the research area assigned by the planner agent.

The system can include more than three scientist agents for hypothesis generation. The scientist agent architecture is scalable, allowing additional scientist agents to be added to analyze additional research areas within the mass spectrometry data. In some cases, the number of scientist agents may be increased to provide more comprehensive coverage of the mass spectrometry data or to enable more specialized analysis of specific compound classes. The scalable architecture enables the system to adapt to datasets of varying complexity and size.

The plurality of scientist agents execute to generate hypotheses based on assigned segments of the mass spectrometry data. Each scientist agent analyzes the assigned data segment with background context provided from the research papers. The scientist agents generate hypotheses with supporting evidence and output the structured hypotheses to the accumulator agent for consolidation. The structured JSON format enables the accumulator agent to process hypotheses from multiple scientist agents and identify duplicate hypotheses for removal.

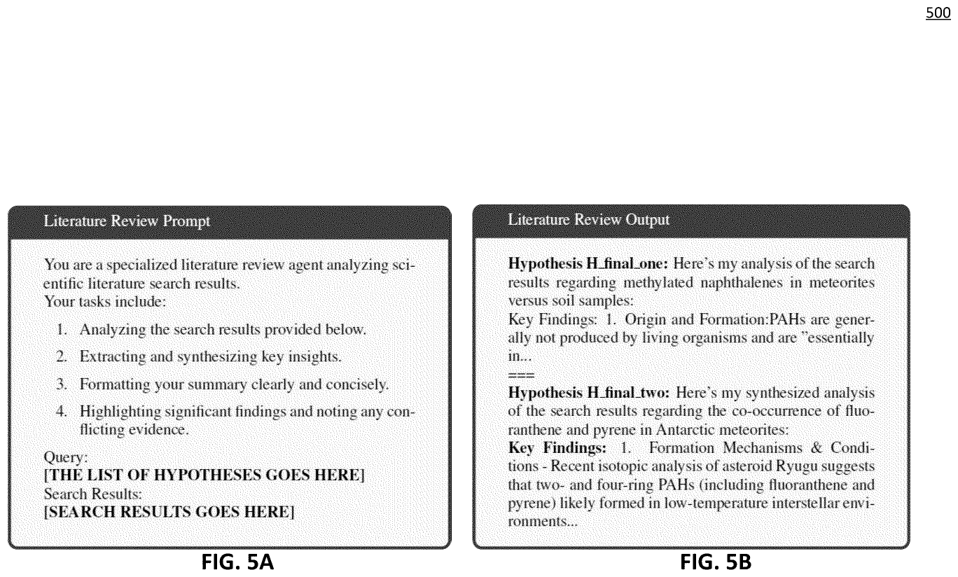

Referring to FIG. 5A and FIG. 5B, a literature review agent prompt interface 500 is illustrated. The literature review agent prompt interface 500 provides a structured prompt template and corresponding output format for the literature review agent 112 to review literature relevant to each of the consolidated hypotheses received from the accumulator agent 110. The literature review agent prompt interface 500 includes a literature review prompt section shown in FIG. 5A and a literature review output section shown in FIG. 5B.

As described previously, the literature review agent 112 receives the consolidated hypotheses from the accumulator agent 110 and reviews literature relevant to each of the consolidated hypotheses. The literature review agent prompt interface 500 describes a specialized literature review agent that analyzes scientific literature search results. The literature review agent prompt interface 500 specifies tasks for the literature review agent 112, including analyzing the search results provided, extracting and synthesizing key insights, formatting summaries clearly and concisely, and highlighting significant findings and noting any conflicting evidence.

With continued reference to FIG. 5A, the literature review agent prompt interface 500 includes a query field where a list of hypotheses is inserted and a search results field where search results are inserted. The literature review agent 112 utilizes Semantic Scholar to locate relevant research papers for each hypothesis. Reviewing literature relevant to each of the consolidated hypotheses comprises searching a scientific literature database for papers corresponding to each hypothesis and synthesizing key insights from search results. The literature review agent 112 retrieves and analyzes up to five pertinent paper snippets per query, enabling comprehensive coverage of relevant literature for each hypothesis.

The literature review agent 112 searches a scientific literature database such as Semantic Scholar for papers corresponding to each consolidated hypothesis. The literature review agent 112 processes the search results by extracting insights from the retrieved papers and synthesizing information across multiple sources. The literature review agent prompt interface 500 instructs the literature review agent 112 to provide a well-organized summary addressing the query, key discoveries, and research gaps, and to include any relevant citations.

As further shown in FIG. 5B, the literature review output section of the literature review agent prompt interface 500 displays example outputs from the literature review agent 112. The literature review output section includes Hypothesis H\_final\_one providing analysis of search results regarding methylated naphthalenes in meteorites versus soil samples. The key findings for Hypothesis H\_final\_one indicate that polycyclic aromatic hydrocarbons are generally not produced by living organisms. The literature review output section also includes Hypothesis H\_final\_two providing synthesized analysis of search results regarding the co-occurrence of fluoranthene and pyrene in Antarctic meteorites.

With continued reference to FIG. 5B, the key findings for Hypothesis H\_final\_two describe formation mechanisms and conditions based on recent isotopic analysis of asteroid Ryugu. The literature review agent 112 synthesizes key insights from search results to indicate that two-ring and four-ring polycyclic aromatic hydrocarbons including fluoranthene and pyrene likely formed in low-temperature interstellar environments. The literature review agent 112 highlights significant findings from the scientific literature and notes any potential conflicts between the generated hypotheses and existing research.

The literature review agent 112 searches for and summarizes literature relevant to each consolidated hypothesis. The literature review agent 112 outputs the literature review summary to the critic agent 114 for evaluation of the consolidated hypotheses. The literature review agent prompt interface 500 enables the literature review agent 112 to provide structured summaries that support the evaluation process performed by the critic agent 114.

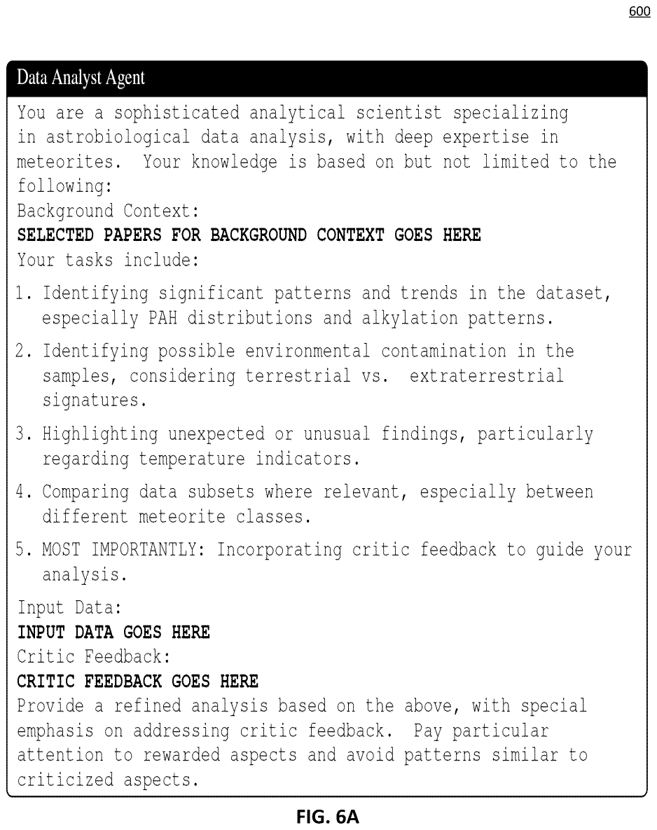

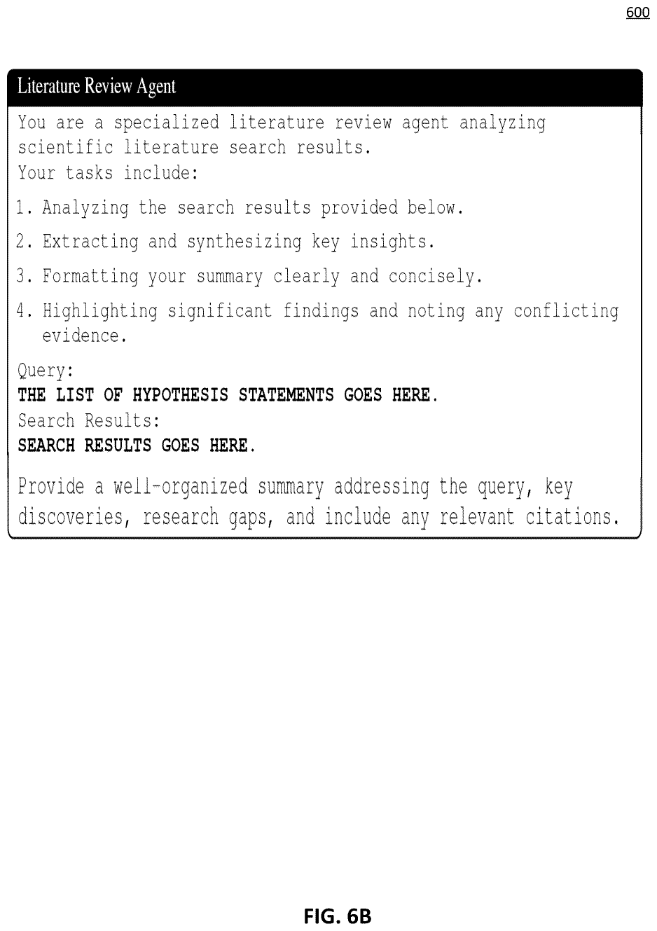

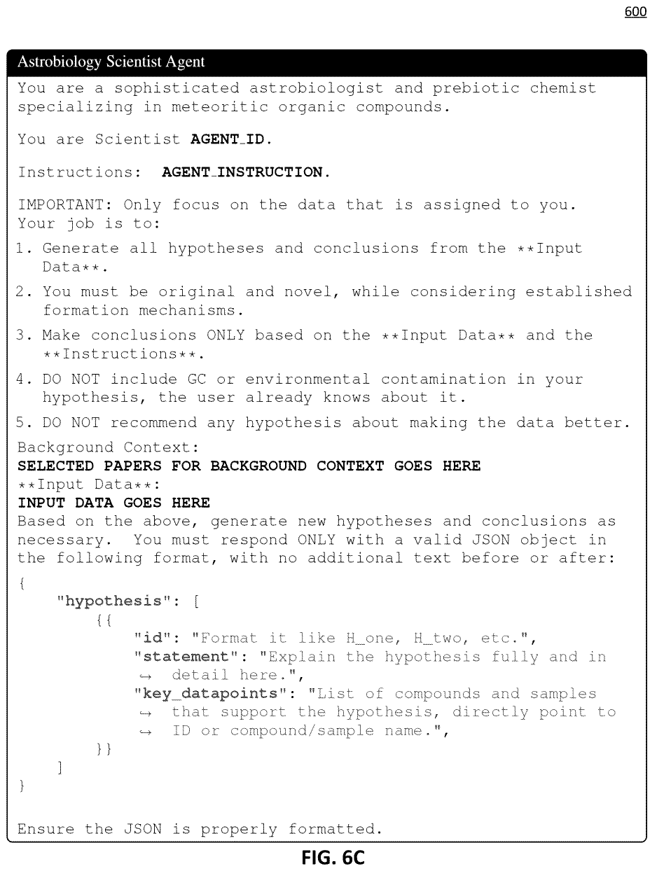

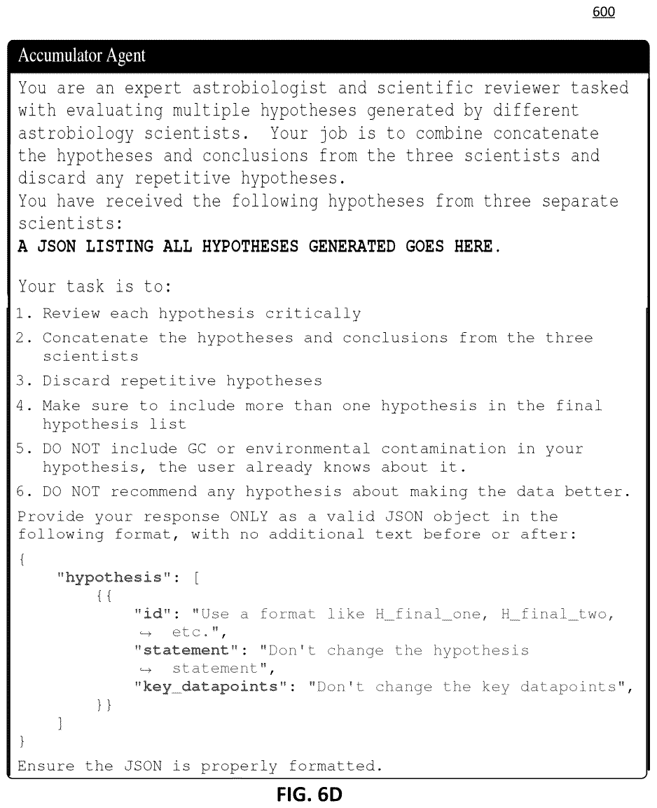

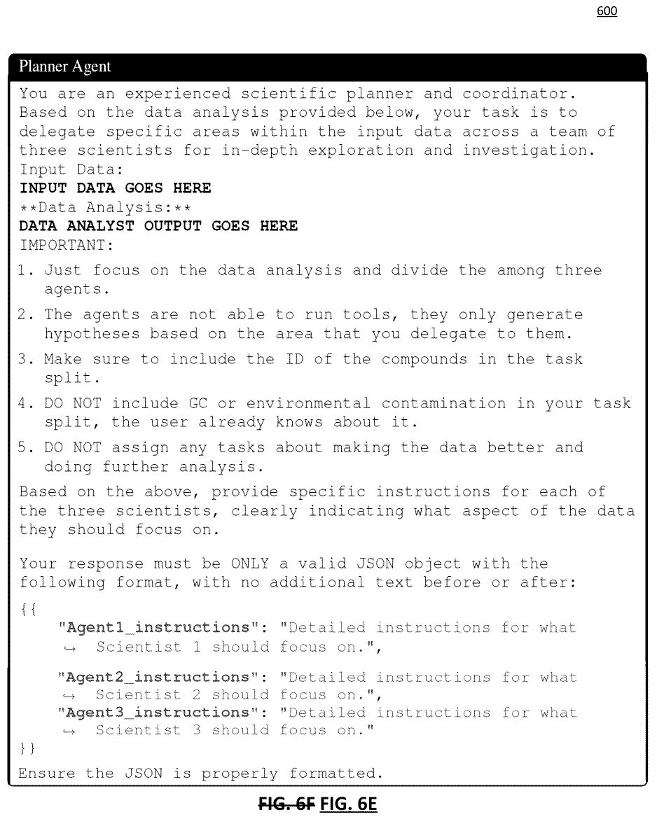

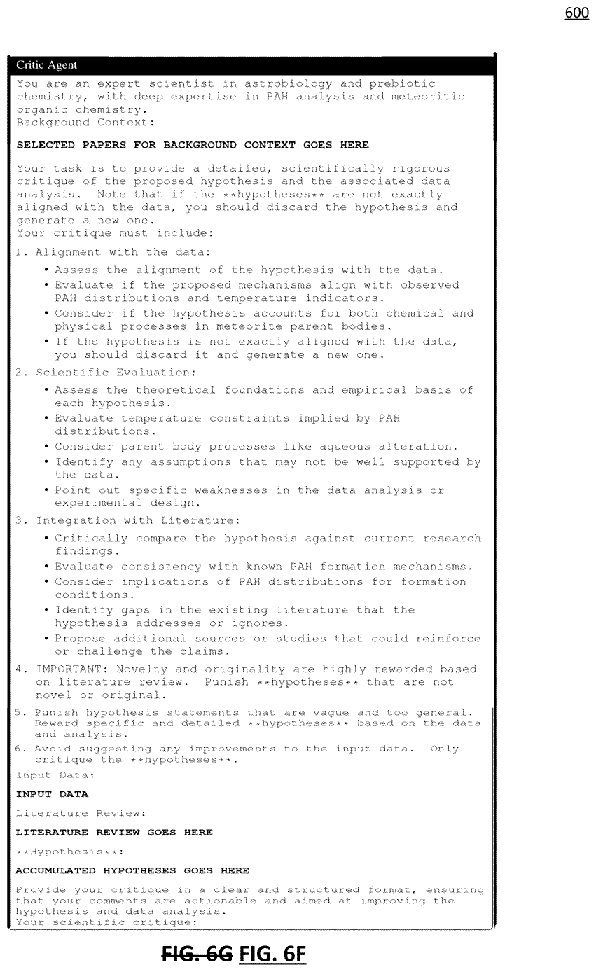

Referring to FIG. 6A, a critic agent prompt interface 600 configured for the data analyst agent 104 is illustrated. The critic agent prompt interface 600 provides a structured prompt template for the data analyst agent 104 to analyze mass spectrometry data with astrobiological expertise. The critic agent prompt interface 600 includes a background context section indicating where selected papers for background context are to be inserted, enabling the data analyst agent 104 to access relevant scientific literature during analysis.

With continued reference to FIG. 6A, the critic agent prompt interface 600 lists tasks for the data analyst agent 104. The tasks include identifying patterns and trends in the dataset with emphasis on polycyclic aromatic hydrocarbon distributions and alkylation patterns, identifying possible environmental contamination in samples while considering terrestrial versus extraterrestrial signatures, highlighting unexpected or unusual findings particularly regarding temperature indicators, comparing data subsets where relevant especially between different meteorite classes, and incorporating critic feedback to guide the analysis. The critic agent prompt interface 600 includes an input data section and a critic feedback section, enabling the data analyst agent 104 to refine its analysis based on feedback from the critic agent 114.

The critic agent prompt interface 600 instructs the data analyst agent 104 to provide a refined analysis with emphasis on addressing critic feedback. The data analyst agent 104 pays attention to rewarded aspects and avoids patterns similar to criticized aspects in subsequent iterations. The critic agent 114 provides feedback to the data analyst agent 104 for iterative refinement of subsequent analyses, as described previously.

Referring to FIG. 6B, the critic agent prompt interface 600 configured for the literature review agent 112 is illustrated. The critic agent prompt interface 600 displays instructions for a specialized literature review agent that analyzes scientific literature search results. The critic agent prompt interface 600 specifies tasks for the literature review agent 112, including analyzing the search results provided, extracting and synthesizing key insights, formatting the summary clearly and concisely, and highlighting significant findings and noting any conflicting evidence.

With continued reference to FIG. 6B, the critic agent prompt interface 600 includes a query section where the list of hypothesis statements is inserted and a search results section where the search results are inserted. The critic agent prompt interface 600 instructs the literature review agent 112 to provide a well-organized summary addressing the query, key discoveries, research gaps, and to include any relevant citations. The literature review agent 112 processes search results to support the evaluation of consolidated hypotheses by the critic agent 114.

Referring to FIG. 6C, the critic agent prompt interface 600 configured for the scientist agents 108 is illustrated. The critic agent prompt interface 600 displays instructions for a sophisticated astrobiologist and prebiotic chemist specializing in meteoritic organic compounds. The critic agent prompt interface 600 identifies each scientist agent with a specific identifier and corresponding instructions. The critic agent prompt interface 600 specifies that each scientist agent focuses on assigned data and outlines tasks including generating hypotheses and conclusions from the input data, being original and novel while considering established formation mechanisms, and making conclusions based on the input data and instructions.

With continued reference to FIG. 6C, the critic agent prompt interface 600 includes a background context section where selected papers are inserted and an input data section where input data is inserted. The critic agent prompt interface 600 specifies that each scientist agent responds with a valid JSON object containing a hypothesis array with entries including an identifier field, a statement field explaining the hypothesis fully and in detail, and a key\_datapoints field listing compounds and samples that support the hypothesis. The structured format enables the accumulator agent 110 to process hypotheses from multiple scientist agents.

Referring to FIG. 6D, the critic agent prompt interface 600 configured for the accumulator agent 110 is illustrated. The critic agent prompt interface 600 displays instructions for an expert astrobiologist and scientific reviewer tasked with combining and deduplicating hypotheses from the scientist agents 108. The accumulator agent 110 consolidates the hypotheses generated by the plurality of scientist agents by discarding duplicate hypotheses. The accumulator agent 110 performs hypothesis deduplication by identifying and consolidating substantially similar hypotheses, ensuring a streamlined and non-redundant set of hypotheses for further investigation.

With continued reference to FIG. 6D, the critic agent prompt interface 600 indicates that a JSON listing of all hypotheses generated is provided as input to the accumulator agent 110. The critic agent prompt interface 600 specifies tasks for the accumulator agent 110, including reviewing each hypothesis critically, concatenating the hypotheses and conclusions from the scientist agents, discarding repetitive hypotheses, and ensuring more than one hypothesis is included in the final hypothesis list. The accumulator agent 110 executes to consolidate the hypotheses by removing duplicate hypotheses, producing a consolidated set of unique hypotheses for evaluation by the critic agent 114.

The critic agent prompt interface 600 specifies that the accumulator agent 110 responds with a valid JSON object containing a hypothesis array with objects including an identifier field, a statement field, and a key\_datapoints field. The JSON format enables the literature review agent 112 and the critic agent 114 to process the consolidated hypotheses in a structured manner.

Referring to FIG. 6E, the critic agent prompt interface 600 configured for the planner agent 106 is illustrated. The critic agent prompt interface 600 displays a planner agent configuration that instructs the planner agent 106 to function as an experienced scientific planner and coordinator. The critic agent prompt interface 600 shows that the planner agent 106 receives input data and data analysis output, and is tasked with delegating specific areas within the input data across a team of scientist agents for in-depth exploration and investigation.

With continued reference to FIG. 6E, the critic agent prompt interface 600 includes guidelines for the planner agent 106, including focusing on data analysis and dividing work among the scientist agents, noting that the scientist agents generate hypotheses based on delegated areas, requiring inclusion of compound identifiers in task splits, and excluding environmental contamination from task splits. The critic agent prompt interface 600 specifies that the planner agent 106 responds with a valid JSON object containing instruction fields for each scientist agent, with no additional text before or after the JSON object.

Referring to FIG. 6F, the critic agent prompt interface 600 configured for hypothesis evaluation is illustrated. The critic agent prompt interface 600 displays instructions for an expert scientist in astrobiology and prebiotic chemistry with expertise in polycyclic aromatic hydrocarbon analysis and meteoritic organic chemistry. The critic agent 114 evaluates the consolidated hypotheses based on the reviewed literature. The critic agent prompt interface 600 includes a background context section indicating where selected papers for background context are inserted.

With continued reference to FIG. 6F, the critic agent prompt interface 600 specifies that the task is to provide a detailed, scientifically rigorous critique of proposed hypotheses and associated data analysis. The critic agent 114 evaluates hypotheses based on consistency with experimental data, scientific rigor, theoretical basis from selected papers, and integration with external literature. Evaluating the consolidated hypotheses comprises assessing alignment of each hypothesis with the mass spectrometry data, evaluating scientific rigor and theoretical basis of each hypothesis, and assessing novelty and specificity of each hypothesis.

The critic agent prompt interface 600 outlines critique requirements including alignment with the data. Assessing alignment of each hypothesis with the mass spectrometry data involves evaluating proposed mechanisms against observed polycyclic aromatic hydrocarbon distributions and temperature indicators, considering chemical and physical processes in meteorite parent bodies, and discarding hypotheses that are not aligned with the data. The critic agent 114 assesses whether each hypothesis accounts for both chemical and physical processes in meteorite parent bodies.

As further shown in FIG. 6F, the critic agent prompt interface 600 specifies scientific evaluation requirements. Evaluating scientific rigor and theoretical basis of each hypothesis includes assessing theoretical foundations and empirical basis, evaluating temperature constraints implied by polycyclic aromatic hydrocarbon distributions, considering parent body processes such as aqueous alteration, identifying unsupported assumptions, and pointing out weaknesses in data analysis or experimental design. The critic agent 114 evaluates the theoretical basis of each hypothesis against the selected papers provided as background context.

The critic agent prompt interface 600 includes integration with literature requirements. The critic agent 114 critically compares hypotheses against current research findings, evaluates consistency with known polycyclic aromatic hydrocarbon formation mechanisms, considers implications of polycyclic aromatic hydrocarbon distributions for formation conditions, identifies literature gaps, and proposes additional sources or studies. The critic agent 114 integrates external literature gathered by the literature review agent 112 into the evaluation process.

With continued reference to FIG. 6F, the critic agent prompt interface 600 indicates that novelty and originality are rewarded based on literature review. The critic agent 114 particularly focuses on assessing the novelty and specificity of the claims. Assessing novelty and specificity of each hypothesis involves evaluating how original each hypothesis is compared to existing literature and determining whether each hypothesis makes specific and detailed claims based on the data and analysis. The critic agent prompt interface 600 specifies that vague and general hypothesis statements are penalized while specific and detailed hypotheses based on data and analysis are rewarded.

The critic agent 114 generates structured feedback for iterative refinement. The critic agent 114 provides structured feedback to guide the next round of hypothesis refinement. The feedback from the critic agent 114 is provided to the data analyst agent 104, enabling iteratively refining the analysis based on feedback from the critic agent 114. The iterative refinement process enhances subsequent analyses and hypothesis generation by incorporating critiques and suggestions for improvement from the critic agent 114.

The critic agent prompt interface 600 includes sections for input data, literature review, and accumulated hypotheses, followed by a prompt for the scientific critique output. The critic agent 114 evaluates the consolidated hypotheses and provides feedback to the data analyst agent 104 for iterative refinement, as described previously. The multi-criteria evaluation performed by the critic agent 114 enables comprehensive assessment of each hypothesis across multiple dimensions including alignment with data, scientific rigor, theoretical basis, novelty, and specificity.

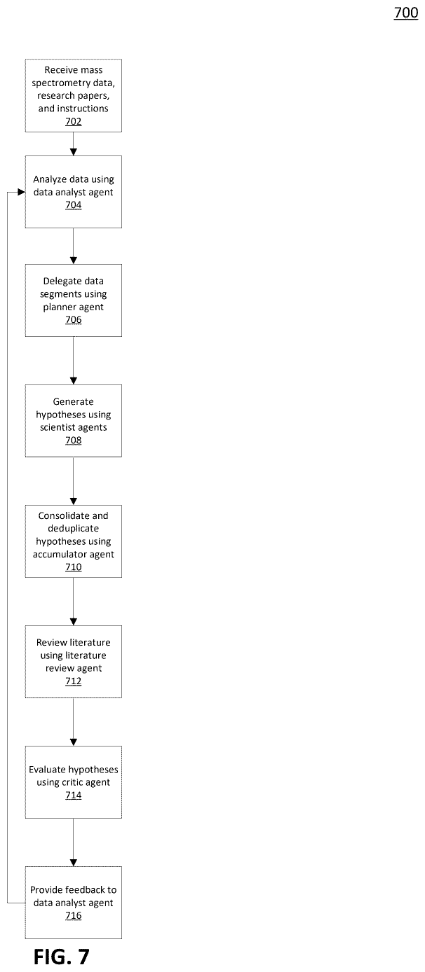

Referring to FIG. 7, a method 700 for generating hypotheses from mass spectrometry data using a multi-agent artificial intelligence system is illustrated. The method 700 provides a structured workflow for receiving input data, processing the input data through multiple specialized agents, and generating hypotheses through an iterative refinement process. The method 700 includes a step 702, a step 704, a step 706, a step 708, a step 710, a step 712, a step 714, and a step 716.

The step 702 comprises receiving mass spectrometry data, research papers, and instructions from a user. The mass spectrometry data may include compound identifiers, mass-to-charge ratios, retention times, compound names, and sample information. The research papers may provide background context for the analysis and hypothesis generation. In some cases, the research papers are converted to a text format and provided as background context to the data analyst agent 104 and the scientist agents 108. The instructions from the user may specify particular areas of focus for the analysis or particular types of hypotheses to be generated.

With continued reference to FIG. 7, the step 704 comprises analyzing the data using the data analyst agent 104. The data analyst agent 104 receives the mass spectrometry data, the research papers, and the instructions from the step 702. The data analyst agent 104 analyzes the mass spectrometry data to identify patterns, unexpected findings, and potential contamination. In some cases, the data analyst agent 104 identifies polycyclic aromatic hydrocarbon distribution patterns in the mass spectrometry data. The data analyst agent 104 may further identify alkylation patterns, contamination indicators, and unique molecular patterns within the mass spectrometry data.

The step 706 comprises delegating data segments to the scientist agents 108 using the planner agent 106. The planner agent 106 receives output from the data analyst agent 104 and delegates segments of the mass spectrometry data to the scientist agents 108 based on the analysis from the data analyst agent 104. The planner agent 106 may assign distinct research focuses to each of the scientist agents 108. In some cases, the planner agent 106 generates instructions for each of the scientist agents 108 in a structured format such as JSON format. The instructions may include compound identifiers to direct each scientist agent to specific compounds for analysis.

As further shown in FIG. 7, the step 708 comprises generating hypotheses using the scientist agents 108. The scientist agents 108 receive instructions and assigned research areas from the planner agent 106. Each of the scientist agents 108 analyzes the assigned data segment with background context provided from the research papers. The scientist agents 108 generate hypotheses based on the delegated segments of the mass spectrometry data. In some cases, generating the hypotheses comprises outputting the hypotheses in a structured format including a hypothesis statement and key data points supporting the hypothesis.

The step 710 comprises consolidating and deduplicating the hypotheses using the accumulator agent 110. The accumulator agent 110 receives hypotheses generated by the scientist agents 108. The accumulator agent 110 consolidates the hypotheses by discarding duplicate hypotheses. The accumulator agent 110 may identify and remove substantially similar hypotheses to produce a consolidated set of unique hypotheses for further evaluation. The consolidated set of unique hypotheses is output to the literature review agent 112.

With continued reference to FIG. 7, the step 712 comprises reviewing literature for each hypothesis using the literature review agent 112. The literature review agent 112 receives the consolidated hypotheses from the accumulator agent 110. The literature review agent 112 searches a scientific literature database for papers corresponding to each hypothesis. In some cases, the literature review agent 112 utilizes Semantic Scholar to locate relevant research papers for each hypothesis. The literature review agent 112 synthesizes key insights from search results and outputs a literature review summary.

The step 714 comprises evaluating the hypotheses using the critic agent 114. The critic agent 114 receives the consolidated hypotheses and the literature review summary from the literature review agent 112. The critic agent 114 evaluates the consolidated hypotheses based on the reviewed literature. In some cases, evaluating the consolidated hypotheses comprises assessing alignment of each hypothesis with the mass spectrometry data, evaluating scientific rigor and theoretical basis of each hypothesis, and assessing novelty and specificity of each hypothesis. The critic agent 114 generates structured feedback and suggestions for improvement.

As further shown in FIG. 7, the step 716 comprises providing feedback from the critic agent 114 to the data analyst agent 104 for iterative refinement of subsequent analyses. A dashed line from the step 716 back to the step 704 indicates an iterative refinement process in which the critic agent feedback is used by the data analyst agent 104 to enhance subsequent analyses and hypothesis generation. The data analyst agent 104 refines its analysis based on the feedback from the critic agent 114, paying attention to rewarded aspects and avoiding patterns similar to criticized aspects in subsequent iterations.

The method 700 may be performed over multiple iterations to improve the quality of generated hypotheses. In each iteration, the feedback from the critic agent 114 guides the data analyst agent 104 to refine the analysis, which in turn affects the delegation of data segments by the planner agent 106 and the hypothesis generation by the scientist agents 108. The iterative refinement process enables the method 700 to produce hypotheses that are increasingly aligned with the mass spectrometry data and consistent with existing scientific literature.

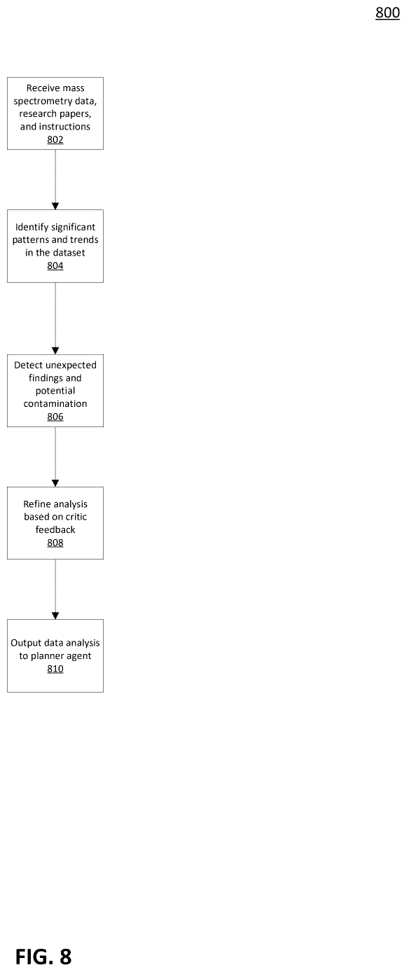

Referring to FIG. 8, a method 800 for analyzing mass spectrometry data using the data analyst agent 104 is illustrated. The method 800 provides a structured workflow for receiving input data, identifying patterns and unexpected findings, refining analysis based on feedback, and outputting data analysis for further processing by the planner agent 106. The method 800 includes a step 802, a step 804, a step 806, a step 808, and a step 810.

The step 802 comprises receiving mass spectrometry data, research papers, and instructions. The data analyst agent 104 receives the mass spectrometry data via the user interface 102. The mass spectrometry data may include compound identifiers, mass-to-charge ratios, retention times, compound names, and sample information. The research papers may be converted to a text format and provided to the data analyst agent 104 as background context for the analysis. The instructions may specify particular areas of focus for the analysis or particular types of patterns to be identified within the mass spectrometry data.

With continued reference to FIG. 8, the step 804 comprises identifying patterns and trends in the dataset. The data analyst agent 104 examines the mass spectrometry data to identify patterns in compound distributions across different samples. In some cases, the data analyst agent 104 identifies polycyclic aromatic hydrocarbon distribution patterns in the mass spectrometry data. The data analyst agent 104 may identify that unsubstituted polycyclic aromatic hydrocarbons such as naphthalene, fluoranthene, and pyrene are predominantly found in meteorite samples. The data analyst agent 104 may further identify alkylation patterns by detecting methylated polycyclic aromatic hydrocarbons in the mass spectrometry data. The polycyclic aromatic hydrocarbon distribution patterns may include methylated polycyclic aromatic hydrocarbons and unsubstituted polycyclic aromatic hydrocarbons.

The step 806 comprises detecting unexpected findings and potential contamination. The data analyst agent 104 highlights unexpected or unusual findings in the mass spectrometry data, particularly regarding temperature indicators. The data analyst agent 104 may identify unique molecular patterns that suggest formation conditions or thermal histories of the samples. The data analyst agent 104 identifies possible environmental contamination in the samples by considering terrestrial versus extraterrestrial signatures. In some cases, the data analyst agent 104 detects that phthalates appear in soil samples and newer meteorite finds, indicating potential terrestrial contamination. The data analyst agent 104 distinguishes between compounds that are likely of extraterrestrial origin and compounds that may represent terrestrial contamination.

As further shown in FIG. 8, the step 808 comprises refining the analysis based on critic feedback. The data analyst agent 104 receives feedback from the critic agent 114 and incorporates the feedback into subsequent analyses. The data analyst agent 104 refines its findings based on the critic feedback, paying attention to rewarded aspects and avoiding patterns similar to criticized aspects. The iterative refinement process enables the data analyst agent 104 to enhance subsequent analyses by incorporating critiques and suggestions for improvement from the critic agent 114. In some cases, the data analyst agent 104 adjusts the focus of the analysis based on the critic feedback to address gaps or weaknesses identified in previous iterations.

The step 810 comprises outputting the data analysis to the planner agent 106. The data analyst agent 104 outputs the identified patterns, unexpected findings, and potential contamination information to the planner agent 106. The planner agent 106 receives the output from the data analyst agent 104 and uses the output to delegate segments of the mass spectrometry data to the scientist agents 108. The data analysis output from the data analyst agent 104 enables the planner agent 106 to identify distinct research areas within the mass spectrometry data and to generate instructions for each of the scientist agents 108.

The method 800 may be performed as part of the iterative refinement process described with reference to the method 700. In each iteration, the data analyst agent 104 receives feedback from the critic agent 114 at the step 808 and refines the analysis accordingly. The refined analysis is then output to the planner agent 106 at the step 810, enabling the planner agent 106 to delegate data segments to the scientist agents 108 based on the refined analysis. The method 800 enables the data analyst agent 104 to provide increasingly refined analyses over multiple iterations, improving the quality of hypotheses generated by the scientist agents 108.

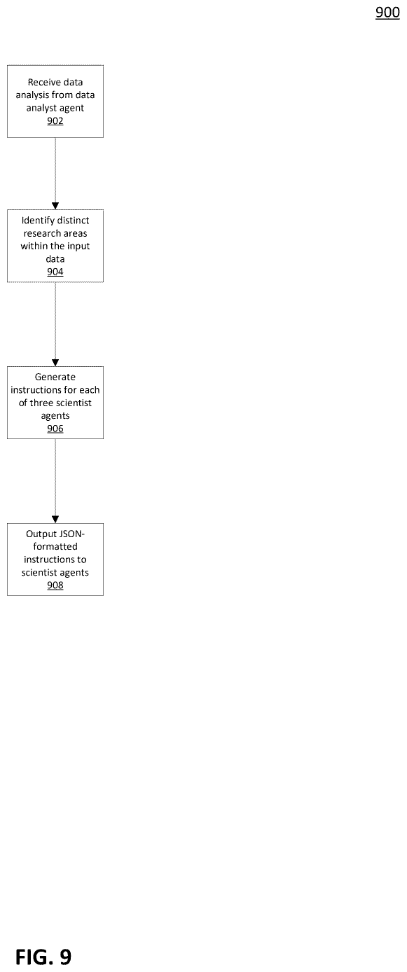

Referring to FIG. 9, a method 900 for delegating data analysis using the planner agent 106 is illustrated. The method 900 provides a structured workflow for receiving data analysis from the data analyst agent 104, identifying distinct research areas within the mass spectrometry data, generating instructions for each of the scientist agents 108, and outputting the instructions in a structured format. The method 900 includes a step 902, a step 904, a step 906, and a step 908.

The step 902 comprises receiving data analysis from the data analyst agent 104. The planner agent 106 receives output from the data analyst agent 104, including identified patterns, unexpected findings, and potential contamination information. The data analysis output from the data analyst agent 104 provides the planner agent 106 with information about compound distributions, polycyclic aromatic hydrocarbon patterns, alkylation patterns, and other characteristics of the mass spectrometry data. The planner agent 106 uses the data analysis output to determine how to delegate segments of the mass spectrometry data to the scientist agents 108.

With continued reference to FIG. 9, the step 904 comprises identifying distinct research areas within the input data. The planner agent 106 analyzes the data analysis output from the data analyst agent 104 to identify distinct research areas that may be assigned to different scientist agents. In some cases, the distinct research areas include polycyclic aromatic hydrocarbon distribution patterns, sulfur-containing and oxygen-containing compounds, and alkylated aromatic compounds. The planner agent 106 may identify additional research areas based on the patterns and findings identified by the data analyst agent 104. The identification of distinct research areas enables the planner agent 106 to divide the analysis work among the scientist agents 108 such that each scientist agent focuses on a specific aspect of the mass spectrometry data.

The step 906 comprises generating instructions for each of the scientist agents 108. The planner agent 106 generates detailed instructions for each scientist agent based on the identified distinct research areas. In some cases, the planner agent 106 generates instructions for three scientist agents, with each scientist agent assigned to analyze a different research area. The instructions may specify particular compound identifiers for each scientist agent to analyze, enabling focused analysis of specific compounds within the mass spectrometry data. The planner agent 106 may assign a first scientist agent to focus on analyzing polycyclic aromatic hydrocarbon distribution patterns, a second scientist agent to investigate sulfur-containing and oxygen-containing compounds, and a third scientist agent to study alkylated aromatic compounds and their derivatives.

As further shown in FIG. 9, the step 908 comprises outputting JSON-formatted instructions to the scientist agents 108. The planner agent 106 outputs the generated instructions in a structured JSON format. The JSON format may include instruction fields for each scientist agent, such as Agent1\_instructions, Agent2\_instructions, and Agent3\ instructions fields. Each instruction field provides detailed instructions for what the respective scientist agent should focus on during hypothesis generation. The structured JSON format enables the scientist agents 108 to receive properly formatted instructions for processing and ensures that each scientist agent receives clear directives for analyzing the assigned data segment.

The method 900 may be performed as part of the method 700 described previously. The planner agent 106 receives data analysis from the data analyst agent 104 at the step 902 following the analysis performed at the step 704 of the method 700. The planner agent 106 then identifies distinct research areas at the step 904, generates instructions at the step 906, and outputs the JSON-formatted instructions at the step 908. The scientist agents 108 receive the instructions from the planner agent 106 and generate hypotheses based on the delegated segments of the mass spectrometry data, as described with reference to the step 708 of the method 700.

The method 900 enables the planner agent 106 to coordinate the hypothesis generation process by distributing analysis tasks among the scientist agents 108. The structured approach of the method 900 allows for in-depth exploration of different aspects of the mass spectrometry data by assigning each scientist agent to a specific research area. The JSON-formatted instructions output at the step 908 provide a standardized format for communicating task assignments to the scientist agents 108, enabling consistent processing of the instructions by each scientist agent.

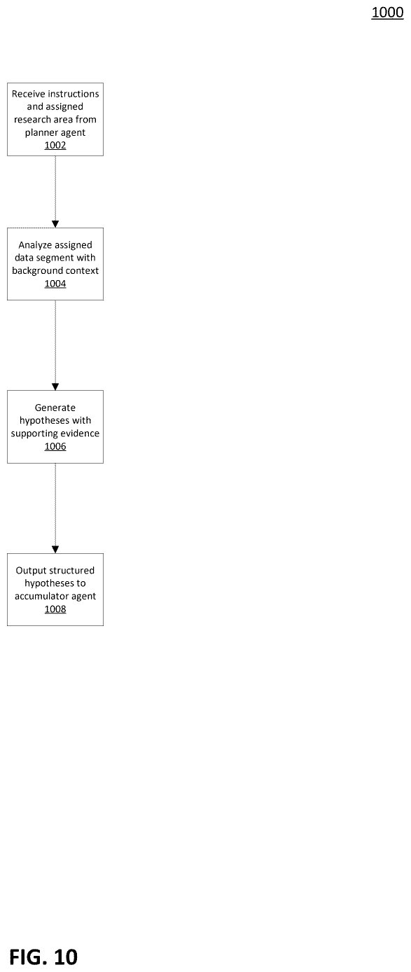

Referring to FIG. 10, a method 1000 for generating hypotheses using hypothesis generation agents is illustrated. The method 1000 provides a structured workflow for receiving instructions from the planner agent 106, analyzing assigned data segments, generating hypotheses with supporting evidence, and outputting structured hypotheses to the accumulator agent 110. The method 1000 includes a step 1002, a step 1004, a step 1006, and a step 1008.

The step 1002 comprises receiving instructions and an assigned research area from the planner agent 106. Each of the scientist agents 108 receives instructions from the planner agent 106 that specify the research area to be analyzed and the compound identifiers to be examined. The instructions may be provided in a structured JSON format, as described previously with reference to the method 900. Each scientist agent receives a distinct research area assignment, enabling focused analysis of specific aspects of the mass spectrometry data. In some cases, the instructions specify particular compound identifiers for each scientist agent to analyze, directing each scientist agent to specific compounds within the mass spectrometry data.

With continued reference to FIG. 10, the step 1004 comprises analyzing the assigned data segment with background context. Each of the scientist agents 108 analyzes the assigned data segment using background context provided from the research papers. The background context may include astrobiological information extracted from academic papers that have been converted to a text format. Each scientist agent examines the compound distributions, molecular patterns, and other characteristics within the assigned data segment. In some cases, each scientist agent considers established formation mechanisms and existing scientific knowledge when analyzing the assigned data segment. The background context enables each scientist agent to interpret the mass spectrometry data within the framework of existing scientific literature.

The step 1006 comprises generating hypotheses with supporting evidence. Each of the scientist agents 108 generates hypotheses based on the analysis of the assigned data segment. Each hypothesis includes a hypothesis statement that explains the hypothesis fully and in detail. Each hypothesis further includes supporting evidence in the form of key data points that substantiate the hypothesis. The key data points may include compound identifiers, sample names, and other information from the mass spectrometry data that support the hypothesis. In some cases, each scientist agent generates multiple hypotheses based on different patterns or findings within the assigned data segment.

As further shown in FIG. 10, the step 1008 comprises outputting structured hypotheses to the accumulator agent 110. Each of the scientist agents 108 outputs the generated hypotheses in a structured format. The structured format may comprise a JSON object containing an identifier, the hypothesis statement, and the key data points, as described previously. The identifier may be formatted using a naming convention such as H\_one, H\_two, or similar designations to distinguish between different hypotheses. The accumulator agent 110 receives the structured hypotheses from each of the scientist agents 108 and consolidates the hypotheses by discarding duplicate hypotheses.

The method 1000 may be performed by each of the scientist agents 108 in parallel. Each scientist agent receives instructions at the step 1002, analyzes the assigned data segment at the step 1004, generates hypotheses at the step 1006, and outputs structured hypotheses at the step 1008. The parallel execution of the method 1000 by multiple scientist agents enables concurrent analysis of different segments of the mass spectrometry data. In some cases, the plurality of scientist agents comprises three scientist agents, each scientist agent configured to analyze a distinct research area within the mass spectrometry data.

The method 1000 may be performed as part of the method 700 described previously. The scientist agents 108 receive instructions from the planner agent 106 at the step 1002 following the delegation performed at the step 706 of the method 700. The scientist agents 108 then analyze the assigned data segments at the step 1004, generate hypotheses at the step 1006, and output structured hypotheses at the step 1008. The accumulator agent 110 receives the structured hypotheses from the scientist agents 108 and consolidates the hypotheses, as described with reference to the step 710 of the method 700.

The method 1000 enables each of the scientist agents 108 to generate hypotheses that are specific to the assigned research area. The structured output format at the step 1008 enables the accumulator agent 110 to process hypotheses from multiple scientist agents and identify duplicate hypotheses for removal. The method 1000 facilitates the generation of hypotheses that are supported by both the mass spectrometry data and the background context provided from the research papers.

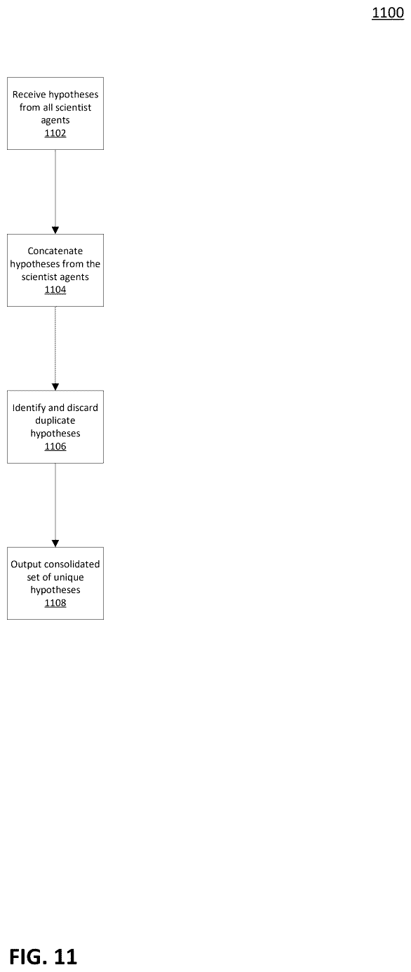

Referring to FIG. 11, a method 1200 for consolidating hypotheses using the accumulator agent 110 is illustrated. The method 1200 provides a structured workflow for receiving hypotheses from the scientist agents 108, concatenating the hypotheses, identifying and discarding duplicate hypotheses, and outputting a consolidated set of unique hypotheses for further evaluation. The method 1200 includes a step 1202, a step 1204, a step 1210, and a step 1300.

The step 1202 comprises receiving hypotheses from all of the scientist agents 108. The accumulator agent 110 receives hypotheses generated by each of the scientist agents 108. As described previously, each of the scientist agents 108 outputs hypotheses in a structured format comprising a JSON object containing an identifier, a hypothesis statement, and key data points. The accumulator agent 110 collects the structured hypotheses from each of the scientist agents 108 for processing. In some cases, the accumulator agent 110 receives hypotheses from three scientist agents, with each scientist agent having analyzed a distinct research area within the mass spectrometry data.

With continued reference to FIG. 11, the step 1204 comprises concatenating hypotheses from the scientist agents 108. The accumulator agent 110 combines the hypotheses received from each of the scientist agents 108 into a single collection. The concatenation process aggregates hypotheses from multiple scientist agents into a unified set for subsequent processing. The accumulator agent 110 may maintain the structured format of each hypothesis during the concatenation process, preserving the identifier, hypothesis statement, and key data points for each hypothesis.

The step 1210 comprises identifying and discarding duplicate hypotheses. The accumulator agent 110 analyzes the concatenated hypotheses to identify substantially similar hypotheses. The accumulator agent 110 performs hypothesis deduplication by comparing hypothesis statements and key data points across the concatenated hypotheses. In some cases, the accumulator agent 110 identifies hypotheses that address the same patterns or findings within the mass spectrometry data and removes redundant hypotheses from the collection. The accumulator agent 110 may consolidate substantially similar hypotheses into a single hypothesis entry, retaining the hypothesis statement and key data points that provide the most comprehensive representation of the underlying finding.

As further shown in FIG. 11, the step 1300 comprises outputting a consolidated set of unique hypotheses. The accumulator agent 110 outputs the deduplicated hypotheses as a consolidated set for further evaluation by the literature review agent 112 and the critic agent 114. The consolidated set of unique hypotheses may be output in a structured JSON format, as described previously with reference to the critic agent prompt interface 600 configured for the accumulator agent 110. The structured format enables the literature review agent 112 to process each hypothesis and search for relevant literature. The consolidated set of unique hypotheses provides a streamlined and non-redundant collection of hypotheses for evaluation, reducing redundancy in the subsequent literature review and evaluation processes.

The method 1200 may be performed as part of the method 700 described previously. The accumulator agent 110 receives hypotheses from the scientist agents 108 at the step 1202 following the hypothesis generation performed at the step 708 of the method 700. The accumulator agent 110 then concatenates the hypotheses at the step 1204, identifies and discards duplicate hypotheses at the step 1210, and outputs the consolidated set of unique hypotheses at the step 1300. The literature review agent 112 receives the consolidated set of unique hypotheses from the accumulator agent 110 and reviews literature relevant to each of the consolidated hypotheses, as described with reference to the step 712 of the method 700.

The method 1200 enables the accumulator agent 110 to reduce redundancy in the hypotheses generated by the scientist agents 108. The deduplication process at the step 1210 ensures that the literature review agent 112 and the critic agent 114 evaluate a non-redundant set of hypotheses, improving the efficiency of the subsequent evaluation processes. The method 1200 facilitates the consolidation of hypotheses from multiple scientist agents into a unified collection that represents the distinct findings and patterns identified within the mass spectrometry data.

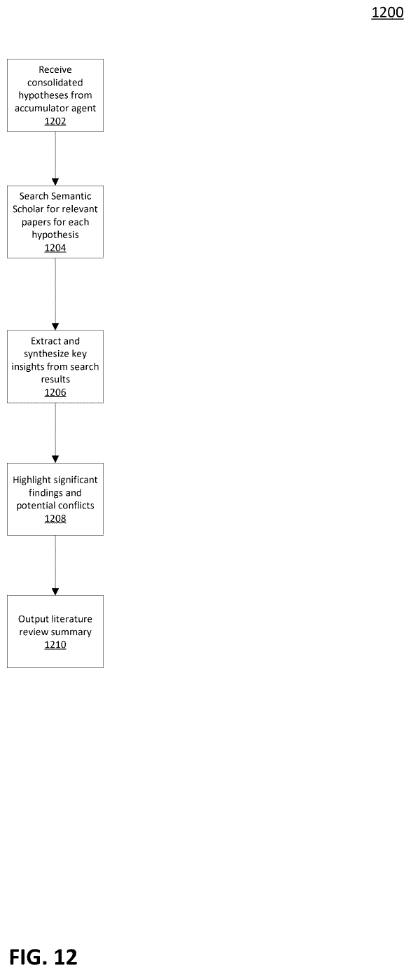

Referring to FIG. 12, a method 1600 for reviewing literature using the literature review agent 112 is illustrated. The method 1600 provides a structured workflow for receiving consolidated hypotheses from the accumulator agent 110, searching for relevant literature, extracting and synthesizing insights from search results, highlighting findings and potential conflicts, and outputting a literature review summary for evaluation by the critic agent 114. The method 1600 includes a step 1602, a step 1604, a step 1606, a step 1608, and a step 1610.

The step 1602 comprises receiving consolidated hypotheses from the accumulator agent 110. The literature review agent 112 receives the consolidated set of unique hypotheses output by the accumulator agent 110 following the deduplication process described previously. The consolidated hypotheses may be provided in a structured JSON format containing an identifier, a hypothesis statement, and key data points for each hypothesis. The literature review agent 112 processes each hypothesis in the consolidated set to identify relevant literature for evaluation. In some cases, the literature review agent 112 extracts the hypothesis statement from each hypothesis entry to formulate search queries for the scientific literature database.

With continued reference to FIG. 12, the step 1604 comprises searching Semantic Scholar for relevant papers for each hypothesis. The literature review agent 112 utilizes Semantic Scholar to locate relevant research papers corresponding to each hypothesis in the consolidated set. The literature review agent 112 formulates search queries based on the hypothesis statements and key data points contained in each hypothesis entry. In some cases, the literature review agent 112 retrieves and analyzes up to five pertinent paper snippets per query, enabling comprehensive coverage of relevant literature for each hypothesis. The search process enables the literature review agent 112 to identify scientific publications that address the subject matter of each hypothesis.

The step 1606 comprises extracting and synthesizing key insights from search results. The literature review agent 112 processes the search results returned from Semantic Scholar by extracting insights from the retrieved papers. The literature review agent 112 synthesizes information across multiple sources to provide a comprehensive summary of the relevant literature for each hypothesis. In some cases, the literature review agent 112 identifies key discoveries, research gaps, and relevant citations from the search results. The synthesis process enables the literature review agent 112 to consolidate findings from multiple papers into a coherent summary that supports the evaluation of each hypothesis.

As further shown in FIG. 12, the step 1608 comprises highlighting significant findings and potential conflicts. The literature review agent 112 identifies significant findings from the scientific literature that support or contradict each hypothesis. The literature review agent 112 notes any conflicting evidence between the generated hypotheses and existing research. In some cases, the literature review agent 112 identifies areas where the hypothesis aligns with established scientific knowledge and areas where the hypothesis diverges from existing literature. The identification of potential conflicts enables the critic agent 114 to evaluate the consistency of each hypothesis with current research findings.

The step 1610 comprises outputting the literature review summary. The literature review agent 112 outputs a well-organized summary addressing each hypothesis, key discoveries from the literature, research gaps, and relevant citations. The literature review summary may be formatted to provide clear and concise information for each hypothesis in the consolidated set. The literature review agent 112 outputs the literature review summary to the critic agent 114 for evaluation of the consolidated hypotheses. In some cases, the literature review summary includes synthesized analysis of search results that describes formation mechanisms, conditions, and other scientific findings relevant to each hypothesis.

The method 1600 may be performed as part of the method 700 described previously. The literature review agent 112 receives consolidated hypotheses from the accumulator agent 110 at the step 1602 following the consolidation performed at the step 710 of the method 700. The literature review agent 112 then searches Semantic Scholar at the step 1604, extracts and synthesizes insights at the step 1606, highlights significant findings and potential conflicts at the step 1608, and outputs the literature review summary at the step 1610. The critic agent 114 receives the literature review summary from the literature review agent 112 and evaluates the consolidated hypotheses, as described with reference to the step 714 of the method 700.

The method 1600 enables the literature review agent 112 to provide literature-based context for each hypothesis generated by the scientist agents 108. The literature review process at the steps 1604, 1606, and 1608 enables the critic agent 114 to evaluate each hypothesis against existing scientific knowledge. The method 1600 facilitates the integration of external literature into the hypothesis evaluation process, enabling comprehensive assessment of each hypothesis based on both the mass spectrometry data and the scientific literature.

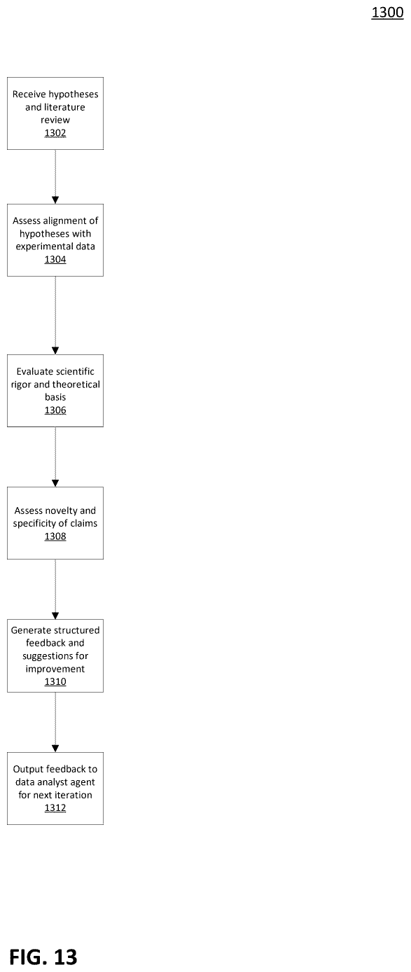

Referring to FIG. 13, a method 1700 for evaluating hypotheses using the critic agent 114 is illustrated. The method 1700 provides a structured workflow for receiving hypotheses and literature review information, assessing alignment with experimental data, evaluating scientific rigor and theoretical basis, assessing novelty and specificity, generating structured feedback, and outputting feedback to the data analyst agent 104 for iterative refinement. The method 1700 includes a step 1702, a step 1704, a step 1706, a step 1708, a step 1710, and a step 1712.

The step 1702 comprises receiving hypotheses and a literature review. The critic agent 114 receives the consolidated hypotheses from the accumulator agent 110 and the literature review summary from the literature review agent 112. The consolidated hypotheses may be provided in a structured JSON format containing an identifier, a hypothesis statement, and key data points for each hypothesis, as described previously. The literature review summary provides literature-based context for each hypothesis, including key discoveries, research gaps, and relevant citations from the scientific literature. The critic agent 114 uses both the consolidated hypotheses and the literature review summary to perform a comprehensive evaluation of each hypothesis.

With continued reference to FIG. 13, the step 1704 comprises assessing alignment of the hypotheses with experimental data. The critic agent 114 evaluates whether each hypothesis aligns with the mass spectrometry data. The critic agent 114 assesses whether the proposed mechanisms in each hypothesis align with observed polycyclic aromatic hydrocarbon distributions and temperature indicators within the mass spectrometry data. In some cases, the critic agent 114 considers whether each hypothesis accounts for both chemical and physical processes in meteorite parent bodies. The critic agent 114 may discard hypotheses that are not aligned with the mass spectrometry data and generate feedback indicating the misalignment.

The step 1706 comprises evaluating scientific rigor and theoretical basis of each hypothesis. The critic agent 114 assesses the theoretical foundations and empirical basis of each hypothesis. The critic agent 114 evaluates temperature constraints implied by polycyclic aromatic hydrocarbon distributions within the mass spectrometry data. In some cases, the critic agent 114 considers parent body processes such as aqueous alteration when evaluating the theoretical basis of each hypothesis. The critic agent 114 identifies assumptions within each hypothesis that may not be well supported by the mass spectrometry data. The critic agent 114 may point out weaknesses in the data analysis or experimental design that affect the scientific rigor of each hypothesis.

As further shown in FIG. 13, the step 1708 comprises assessing novelty and specificity of the claims in each hypothesis. The critic agent 114 evaluates how original each hypothesis is compared to existing literature identified by the literature review agent 112. The critic agent 114 determines whether each hypothesis makes specific and detailed claims based on the mass spectrometry data and the analysis performed by the scientist agents 108. In some cases, the critic agent 114 penalizes vague and general hypothesis statements while rewarding specific and detailed hypotheses based on the data and analysis. The assessment of novelty and specificity enables the critic agent 114 to distinguish between hypotheses that provide new insights and hypotheses that merely restate existing knowledge.

The step 1710 comprises generating structured feedback and suggestions for improvement. The critic agent 114 generates feedback based on the assessments performed at the step 1704, the step 1706, and the step 1708. The structured feedback may include critiques of each hypothesis, suggestions for improving the alignment of hypotheses with the mass spectrometry data, and recommendations for enhancing the scientific rigor and novelty of the hypotheses. In some cases, the critic agent 114 identifies aspects of the analysis that should be rewarded in subsequent iterations and patterns that should be avoided. The structured feedback provides guidance for the next round of hypothesis refinement.

With continued reference to FIG. 13, the step 1712 comprises outputting feedback to the data analyst agent 104 for the next iteration. The critic agent 114 provides the structured feedback to the data analyst agent 104, enabling iterative refinement of subsequent analyses. The feedback from the critic agent 114 is used by the data analyst agent 104 to enhance subsequent analyses and hypothesis generation, as described previously with reference to the method 700. The data analyst agent 104 refines its analysis based on the feedback from the critic agent 114, paying attention to rewarded aspects and avoiding patterns similar to criticized aspects in subsequent iterations.

The method 1700 may be performed as part of the method 700 described previously. The critic agent 114 receives hypotheses and the literature review at the step 1702 following the literature review performed at the step 712 of the method 700. The critic agent 114 then assesses alignment with experimental data at the step 1704, evaluates scientific rigor and theoretical basis at the step 1706, assesses novelty and specificity at the step 1708, generates structured feedback at the step 1710, and outputs feedback to the data analyst agent 104 at the step 1712. The feedback output at the step 1712 corresponds to the feedback provided at the step 716 of the method 700, enabling the iterative refinement process.

The method 1700 enables the critic agent 114 to perform multi-criteria evaluation of each hypothesis across multiple dimensions. The evaluation performed at the step 1704, the step 1706, and the step 1708 enables comprehensive assessment of each hypothesis based on alignment with data, scientific rigor, theoretical basis, novelty, and specificity. The method 1700 facilitates the iterative refinement process by providing structured feedback that guides the data analyst agent 104 in subsequent iterations, improving the quality of generated hypotheses over multiple iterations.

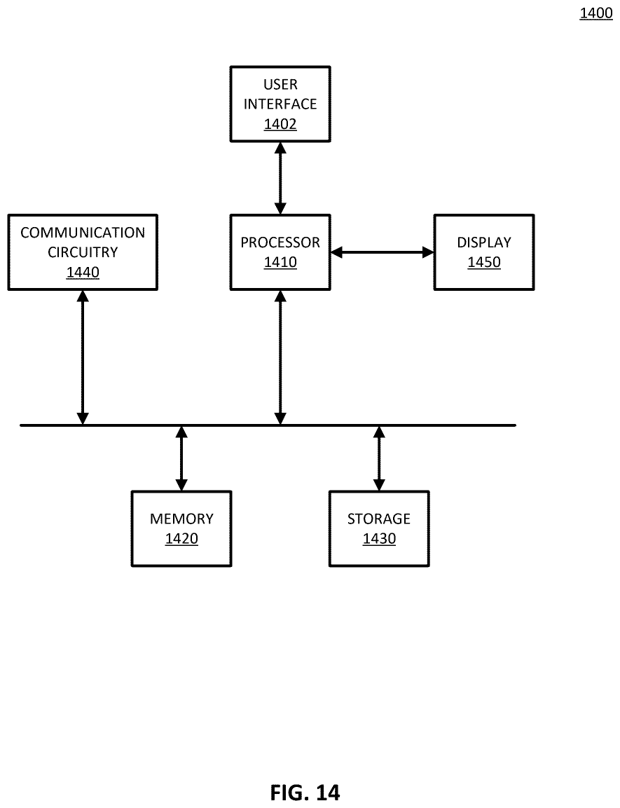

Referring to FIG. 14, a computing device 1000 for implementing the multi-agent artificial intelligence system for hypotheses generation from mass spectrometry data is illustrated. The computing device 1000 includes a processor 1010, a memory 1020 coupled to the processor 1010, a storage 1030 coupled to the processor 1010, communication circuitry 1040, and a display 1050. The computing device 1000 may execute the multi-agent artificial intelligence system including the data analyst agent 104, the planner agent 106, the scientist agents 108, the accumulator agent 110, the literature review agent 112, and the critic agent 114, as described previously.

With continued reference to FIG. 14, the processor 1010 executes instructions to carry out or control the operation of functions performed by the computing device 1000. The processor 1010 may drive the display 1050 and receive user input from a user interface 1002. The user interface 1002 may allow a user to interact with the computing device 1000. The user interface 1002 may take a variety of forms, such as a button, keypad, dial, click wheel, keyboard, display screen, or touch screen. In some cases, the user interface 1002 receives mass spectrometry data, one or more research papers, and instructions from a user, as described previously with reference to the user interface 102.

The processor 1010 may be a system-on-chip such as those found in mobile devices and may include a dedicated Graphics Processing Unit. The processor 1010 may be based on Reduced Instruction-Set Computer architectures or Complex Instruction-Set Computer architectures or any other suitable architecture. In some cases, the processor 1010 includes one or more processing cores. The processor 1010 executes the multi-agent artificial intelligence system by processing instructions stored in the storage 1030 and utilizing the memory 1020 for temporary data storage during execution.

As further shown in FIG. 14, the memory 1020 may include one or more different types of media used by the processor 1010 to perform device functions. The memory 1020 may include memory cache, Read-Only Memory, and Random-Access Memory. The memory 1020 provides temporary storage for data and instructions during processing operations performed by the processor 1010. In some cases, the memory 1020 stores intermediate results generated by the data analyst agent 104, the planner agent 106, the scientist agents 108, the accumulator agent 110, the literature review agent 112, and the critic agent 114 during hypothesis generation.

The storage 1030 may store media, computer program instructions or software, preference information, device profile information, and any other suitable data. The storage 1030 may include one or more non-transitory storage mediums including magnetic disks, both fixed and removable, and tape. The storage 1030 may further include optical media such as Compact Disc-Read Only Memorys and Digital Video Disks. The storage 1030 may also include semiconductor memory devices such as Electrically Programmable Read-Only Memory and Electrically Erasable Programmable Read-Only Memory. The storage 1030 stores instructions that, when executed by the processor 1010, cause the processor 1010 to execute the data analyst agent 104, the planner agent 106, the scientist agents 108, the accumulator agent 110, the literature review agent 112, and the critic agent 114.

With continued reference to FIG. 14, the memory 1020 and the storage 1030 may be used to tangibly retain computer program instructions or code organized into one or more modules and written in any desired computer programming language. When executed by the processor 1010, such computer program code may implement one or more of the methods described herein, including the method 700, the method 800, the method 900, the method 1000, the method 1200, the method 1600, and the method 1700.

The communication circuitry 1040 enables the computing device 1000 to communicate with external systems and networks. The communication circuitry 1040 may enable the literature review agent 112 to access Semantic Scholar for literature searches, as described previously. The display 1050 presents the generated hypotheses, data analysis results, and other outputs to the user. In some cases, the display 1050 displays the user interface 1002 for receiving input from the user.

FIG. 14 further illustrates a processor 1100, a memory 1102, a storage 1104, communication circuitry 1106, and a display 1108. The processor 1100, the memory 1102, the storage 1104, the communication circuitry 1106, and the display 1108 may be configured similarly to the processor 1010, the memory 1020, the storage 1030, the communication circuitry 1040, and the display 1050, respectively. In some cases, the processor 1100, the memory 1102, the storage 1104, the communication circuitry 1106, and the display 1108 represent components of a server or networked computing system that executes the multi-agent artificial intelligence system. The storage 1104 stores instructions that, when executed by the processor 1100, cause the processor 1100 to perform operations comprising receiving mass spectrometry data and research papers, and executing the agents of the method 100 to generate hypotheses.

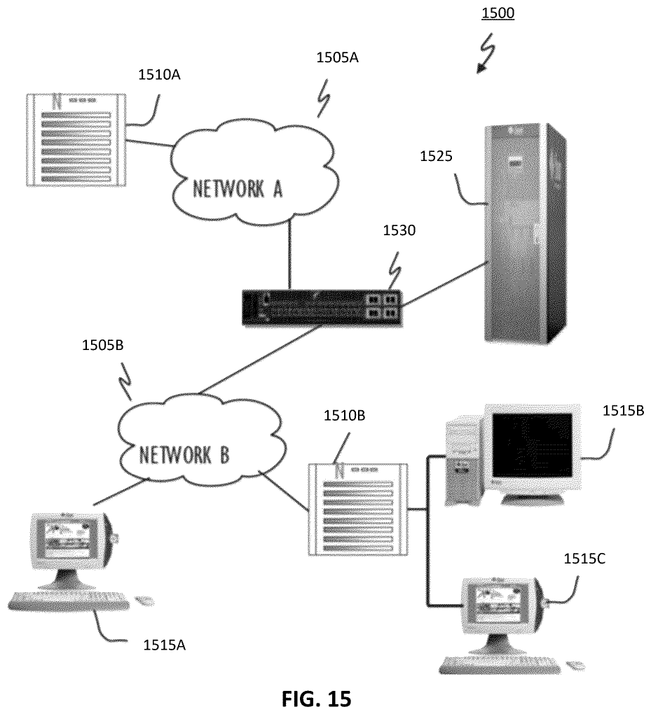

Referring to FIG. 15, a networked computing system 1100 for implementing the multi-agent artificial intelligence system for hypotheses generation from mass spectrometry data is illustrated. The networked computing system 1100 includes a server 1125, a first network 1110A, a second network 1110B, a first client device 1115A, a second client device 1115B, a third client device 1115C, and a network switch 1130. The networked computing system 1100 enables distributed processing for the multi-agent artificial intelligence system by connecting multiple client devices to the server 1125 through the first network 1110A and the second network 1110B.

With continued reference to FIG. 15, the server 1125 is connected to the first network 1110A via a third communication link 1530. The server 1125 processes mass spectrometry data and coordinates the multi-agent artificial intelligence system for hypothesis generation. The server 1125 may execute the data analyst agent 104, the planner agent 106, the scientist agents 108, the accumulator agent 110, the literature review agent 112, and the critic agent 114, as described previously. In some cases, the server 1125 receives mass spectrometry data and research papers from the client devices and returns generated hypotheses and analysis results to the client devices.

The first network 1110A is labeled as NETWORK A and includes the network switch 1130. A first communication link 1105A connects a document or data source to the first network 1110A, enabling data transfer to the networked computing system 1100. The first communication link 1105A may transfer mass spectrometry data, research papers, and instructions from external sources to the first network 1110A for processing by the server 1125. The network switch 1130 routes data between devices connected to the first network 1110A and facilitates communication between the first network 1110A and the second network 1110B.

As further shown in FIG. 15, the second network 1110B is labeled as NETWORK B and is connected to the first network 1110A through the network infrastructure. A second communication link 1105B connects the second network 1110B to additional components within the networked computing system 1100. The second communication link 1105B enables data transfer between the second network 1110B and devices connected to the networked computing system 1100. The first network 1110A and the second network 1110B may operate as separate network segments that are interconnected to enable communication between client devices and the server 1125.

With continued reference to FIG. 15, the first client device 1115A is connected to the second network 1110B. The first client device 1115A may include a display and keyboard configuration for user interaction. The first client device 1115A may provide the user interface 102 for inputting mass spectrometry data, research papers, and instructions to the multi-agent artificial intelligence system. In some cases, the first client device 1115A displays generated hypotheses and analysis results received from the server 1125.

The second client device 1115B is connected to the server 1125. The second client device 1115B may include a monitor and computer tower configuration. The second client device 1115B may provide direct access to the server 1125 for administering the multi-agent artificial intelligence system. In some cases, the second client device 1115B is used to configure the data analyst agent 104, the planner agent 106, the scientist agents 108, the accumulator agent 110, the literature review agent 112, and the critic agent 114.

As further shown in FIG. 15, the third client device 1115C is connected to the second client device 1115B. The third client device 1115C may include a monitor display for viewing results from the multi-agent artificial intelligence system. The third client device 1115C may receive generated hypotheses and analysis results through the connection to the second client device 1115B. In some cases, the third client device 1115C provides an additional viewing station for users to review hypotheses generated by the scientist agents 108.

FIG. 15 further illustrates a server 1312, a first network 1302, a second network 1304, a first client device 1306, a second client device 1308, a third client device 1310, a first communication link 1206, a second communication link 1208, and the third communication link 1530. The server 1312, the first network 1302, the second network 1304, the first client device 1306, the second client device 1308, the third client device 1310, the first communication link 1206, and the second communication link 1208 may be configured similarly to the server 1125, the first network 1110A, the second network 1110B, the first client device 1115A, the second client device 1115B, the third client device 1115C, the first communication link 1105A, and the second communication link 1105B, respectively.

With continued reference to FIG. 15, the networked computing system 1100 topology includes elements of both star and hierarchical configurations. The server 1125 serves as a central processing component for the multi-agent artificial intelligence system. The first network 1110A and the second network 1110B facilitate communication between the client devices 1115A, 1115B, 1115C and the server 1125. Data flows from document sources through the first communication link 1105A to the first network 1110A, then through the third communication link 1530 to the server 1125 for processing, with results distributed back through the network infrastructure to the connected client devices.

The network architecture of the networked computing system 1100 supports distributed processing for the multi-agent artificial intelligence system. In some cases, the server 1125 executes the data analyst agent 104, the planner agent 106, and the critic agent 114, while the scientist agents 108 execute on distributed computing resources connected to the first network 1110A or the second network 1110B. The distributed architecture enables concurrent analysis of different segments of the mass spectrometry data by the scientist agents 108, as described previously with reference to the method 1000.

The client devices 1115A, 1115B, 1115C provide user interfaces for inputting data, viewing results, and interacting with the hypothesis generation system. In some cases, users access the multi-agent artificial intelligence system through the first client device 1115A connected to the second network 1110B, while administrators manage the system through the second client device 1115B connected directly to the server 1125. The network architecture enables multiple users to access the multi-agent artificial intelligence system concurrently through the connected client devices.

The networked computing system 1100 enables the literature review agent 112 to access external scientific literature databases through the communication infrastructure. The server 1125 may communicate with Semantic Scholar through the first network 1110A and the first communication link 1105A to retrieve relevant research papers for each hypothesis, as described previously with reference to the method 1600. The network architecture supports the iterative refinement process by enabling communication between the critic agent 114 and the data analyst agent 104 across the networked computing system 1100.

The multi-agent artificial intelligence system receives and processes input data from users to enable hypothesis generation from mass spectrometry data. The input data processing includes receiving research papers and mass spectrometry data, converting the research papers to a text format, and providing the converted text format as background context to the agents within the system.

The one or more research papers are converted to a text format and provided as background context to the data analyst agent and the plurality of scientist agents. In some cases, the research papers are converted into Markdown format using the pymupdf4llm Python package. The pymupdf4llm Python package processes each research paper and generates text output that is used as background knowledge for the data analyst agent and the scientist agents. The conversion of research papers to Markdown format enables the agents to access the content of the research papers in a format suitable for processing by large language models. The converted text format provides astrobiological context extracted from academic papers that informs the analysis and hypothesis generation performed by the agents.

Users input mass spectrometry data in a LaTeX-formatted table detailing identified compounds and corresponding samples. The LaTeX-formatted table may include compound identifiers, compound names, and sample information indicating which samples contain each compound. The LaTeX table input format provides a structured representation of the mass spectrometry data that enables the data analyst agent to process the data and identify patterns within the dataset.

The mass spectrometry data includes retention times, mass-to-charge ratios, and samples in which compounds were detected. The retention times indicate the time at which each compound elutes from the chromatographic column during analysis. The mass-to-charge ratios provide information about the molecular mass of each compound divided by its charge state. The samples in which compounds were detected indicate the distribution of each compound across the analyzed samples, enabling comparison of compound distributions between different sample types such as meteoritic samples and terrestrial samples.

The mass spectrometry data comprises data obtained from two-dimensional gas chromatography coupled with high-resolution mass spectrometry. In some cases, the system employs two-dimensional gas chromatography coupled with high-resolution time-of-flight mass spectrometry, referred to as GC×GC-HRTOF-MS. The two-dimensional gas chromatography provides enhanced separation of complex mixtures by utilizing two chromatographic columns with different separation characteristics. The high-resolution time-of-flight mass spectrometry provides accurate mass measurements that enable identification of compounds within the samples. The combination of two-dimensional gas chromatography with high-resolution time-of-flight mass spectrometry produces a list of compounds along with peak information including retention times and mass-to-charge ratios.

As described previously, the instructions stored in the storage cause the processor to convert the one or more research papers to a text format and provide the text format as background context to the data analyst agent and the plurality of scientist agents. The operations performed by the processor further comprise converting the research papers to a text format and providing the text format as background context to the data analyst agent and the plurality of scientist agents. The background context derived from the converted research papers enables the agents to interpret the mass spectrometry data within the framework of existing scientific literature and to generate hypotheses that are informed by prior research in the field.

The multi-agent artificial intelligence system may utilize various large language models to power the agents within the system. In some cases, the system uses Claude 3.5 Sonnet as the large language model for the agents. In other cases, the system uses Gemini 2.0 Flash as the large language model for the agents. The selection of a particular large language model may affect the performance characteristics of the multi-agent system, including the quality of generated hypotheses, the consistency of agent collaboration, and the capacity for processing contextual information.

Claude 3.5 Sonnet may be selected for its proven cooperation ability, which is beneficial for effective multi-agent collaboration. The cooperation ability of a large language model refers to the capacity of the model to work effectively with other agents in a multi-agent system, maintaining consistent reasoning and producing outputs that integrate well with outputs from other agents. In some cases, Claude 3.5 Sonnet demonstrates fewer logical errors and greater consistency with scientific literature when generating hypotheses. The cooperation ability of Claude 3.5 Sonnet may enable the data analyst agent, the planner agent, the scientist agents, the accumulator agent, the literature review agent, and the critic agent to collaborate effectively throughout the hypothesis generation workflow.

Gemini 2.0 Flash may be chosen for its extended context window of up to 1M tokens. The extended context window enables the inclusion of a more comprehensive astrobiological context derived from a wide array of scientific literature. In some cases, Gemini 2.0 Flash receives not only research papers but also complete books as background context, taking advantage of the large input token capacity. The extended context window of Gemini 2.0 Flash may enable the agents to access more extensive background knowledge during analysis and hypothesis generation.

The selection of a large language model for the multi-agent system involves trade-offs between cooperative ability and extended contextual input capacity. A large language model with stronger agent collaboration capabilities may produce hypotheses that are more consistent and exhibit fewer logical errors. A large language model with a larger context window may facilitate more creative connections across broader knowledge bases by enabling access to more extensive background information. In some cases, a large language model with superior cooperation ability achieves higher average evaluation scores for consistency and clarity, while a large language model with an extended context window achieves higher novelty scores by generating more original hypotheses.

The multi-agent artificial intelligence system may be configured to use different large language models based on the requirements of a particular analysis task. In some cases, the system is configured to use Claude 3.5 Sonnet when consistency and alignment with existing literature are prioritized. In other cases, the system is configured to use Gemini 2.0 Flash when novelty and access to extensive background context are prioritized. The flexibility to select different large language models enables the multi-agent system to be adapted for different hypothesis generation objectives.

The multi-agent artificial intelligence system processes data from eight meteoric samples and ten terrestrial samples. The meteoric samples and terrestrial samples are systematically analyzed to assess differences in the molecular distributions of organic compounds between the sample types. The analysis of meteoric samples and terrestrial samples enables the identification of patterns that distinguish extraterrestrial organic matter from terrestrial organic matter, supporting hypothesis generation regarding the origins of organic compounds in the samples.

The analysis produces a list of 48 compounds along with peak information for each compound. The peak information includes retention times, mass-to-charge ratios, and the samples in which each compound was detected. The list of 48 compounds provides the input data for the data analyst agent, the planner agent, and the scientist agents to analyze during hypothesis generation. The compound list enables the agents to identify patterns in compound distributions across the meteoric samples and terrestrial samples.

The multi-agent artificial intelligence system conducts experiments over 10 iterations for hypothesis generation. In each iteration, the data analyst agent analyzes the mass spectrometry data, the planner agent delegates data segments to the scientist agents, the scientist agents generate hypotheses, the accumulator agent consolidates the hypotheses, the literature review agent reviews relevant literature, and the critic agent evaluates the hypotheses and provides feedback to the data analyst agent. The 10 iterations enable iterative refinement of the analysis and hypothesis generation, with each iteration incorporating feedback from the critic agent to enhance subsequent analyses.

The total cost of all experiments was less than $100. The cost includes computational resources for executing the large language models that power the agents within the multi-agent artificial intelligence system. The sub-$100 experiment cost demonstrates that the multi-agent artificial intelligence system provides a cost-effective approach for hypothesis generation from mass spectrometry data.

Hypotheses generated by the multi-agent artificial intelligence system are evaluated using six criteria. The six criteria include novelty, consistency with existing knowledge, clarity and precision, empirical support, scope and generalizability, and predictive power. The six evaluation criteria provide a comprehensive framework for assessing the quality of each hypothesis generated by the scientist agents.

Novelty measures how original a hypothesis is compared to existing literature. A hypothesis with high novelty presents findings or interpretations that have not been previously reported in the scientific literature. Consistency with existing knowledge measures whether a hypothesis aligns with established research in the relevant scientific field. A hypothesis with high consistency with existing knowledge is supported by prior findings and does not contradict well-established scientific principles.

Clarity and precision measures whether a hypothesis is clearly stated, specific, and unambiguous. A hypothesis with high clarity and precision presents a well-defined claim that can be understood without ambiguity. Empirical support measures the extent to which the mass spectrometry data support the hypothesis. A hypothesis with high empirical support is directly substantiated by the compound distributions and patterns identified within the mass spectrometry data.

Scope and generalizability measures whether a hypothesis can explain broader phenomena or be applied to wider contexts beyond the specific samples analyzed. A hypothesis with high scope and generalizability has implications that extend beyond the immediate dataset. Predictive power measures whether a hypothesis makes clear, testable predictions. A hypothesis with high predictive power enables the design of experiments or analyses that could confirm or refute the hypothesis.

Each evaluation criterion is rated on a scale from 0 to 10. A score of 0 signifies a complete lack of the quality being measured. A score of 10 represents the highest possible level of the quality being measured. The 0 to 10 rating scale enables quantitative assessment of each hypothesis across the six evaluation criteria, facilitating comparison of hypotheses and identification of hypotheses that meet quality thresholds.

A hypothesis is considered novel if its novelty score is greater than or equal to 5. The novelty threshold of 5 on the 0 to 10 scale indicates that a hypothesis presents findings or interpretations that are at least moderately original compared to existing literature. Hypotheses with novelty scores below 5 may restate existing knowledge without providing new insights.

A hypothesis is considered plausible if the average scores of the other criteria are greater than or equal to 8. The other criteria include consistency with existing knowledge, clarity and precision, empirical support, scope and generalizability, and predictive power. The plausibility threshold of 8 on the 0 to 10 scale indicates that a hypothesis meets a high standard across the non-novelty evaluation criteria. A plausible hypothesis is well-supported by the mass spectrometry data, consistent with existing scientific knowledge, clearly stated, generalizable, and predictive. In some cases, a hypothesis may be both novel and plausible, indicating that the hypothesis presents original findings that are also well-supported and scientifically rigorous.

The multi-agent artificial intelligence system provides applications for analyzing mass spectrometry data from various sources including meteoritic samples and terrestrial samples. The system generates hypotheses related to the origin of life, enhancing scientific discovery by enabling researchers to identify patterns and relationships within complex datasets that may not be apparent through manual analysis. The hypothesis generation capabilities of the system support research into fundamental questions in astrobiology, including how life emerged on Earth and whether organic compounds in meteoritic samples share common origins with terrestrial organic matter.

The multi-agent artificial intelligence system addresses challenges associated with analyzing mass spectrometry data in astrobiology research. Mass spectrometry data from sample return missions and meteoritic samples may contain traces of organic compounds that provide information about prebiotic chemistry and the molecular building blocks that may have contributed to the emergence of life. The system enables researchers to analyze such data within the context of existing scientific literature and to generate plausible hypotheses regarding the formation and distribution of organic compounds across different sample types.

As described previously, analyzing, via a data analyst agent, the mass spectrometry data to identify patterns enables the system to detect compound distributions, alkylation patterns, and other molecular signatures within the dataset. The pattern identification performed by the data analyst agent provides the foundation for subsequent hypothesis generation by the scientist agents. The data analyst agent examines polycyclic aromatic hydrocarbon distributions, sulfur-containing compounds, and other molecular classes to identify characteristics that distinguish meteoritic samples from terrestrial samples.

The components of the multi-agent artificial intelligence system interact to perform the overall function of hypothesis generation from mass spectrometry data through iterative refinement between the agents. The data analyst agent receives mass spectrometry data and research papers, analyzes the data to identify patterns and unexpected findings, and outputs the analysis to the planner agent. The planner agent receives the data analysis and delegates segments of the mass spectrometry data to the scientist agents based on the identified patterns and research areas. Each scientist agent analyzes the assigned data segment and generates hypotheses with supporting evidence.

The accumulator agent receives hypotheses from the scientist agents and consolidates the hypotheses by removing duplicate entries. The literature review agent receives the consolidated hypotheses and searches scientific literature databases to identify relevant research papers for each hypothesis. The critic agent receives the consolidated hypotheses and the literature review summary, evaluates each hypothesis based on alignment with the mass spectrometry data, scientific rigor, and novelty, and provides feedback to the data analyst agent. The feedback from the critic agent enables the data analyst agent to refine subsequent analyses, creating an iterative refinement process that improves the quality of generated hypotheses over multiple iterations.

The iterative refinement process enables the multi-agent artificial intelligence system to produce hypotheses that are increasingly aligned with the mass spectrometry data and consistent with existing scientific literature. In each iteration, the critic agent identifies aspects of the analysis and hypothesis generation that should be enhanced and patterns that should be avoided. The data analyst agent incorporates the feedback from the critic agent into subsequent analyses, adjusting the focus of the analysis to address gaps or weaknesses identified in previous iterations. The iterative approach enables the system to converge toward hypotheses that meet quality thresholds for novelty, consistency with existing knowledge, clarity and precision, empirical support, scope and generalizability, and predictive power.

Table 1 presents selected high-scoring hypotheses generated by the multi-agent artificial intelligence system that received high ratings from astrobiology experts. Each hypothesis in Table 1 is accompanied by the key data points that the system identified as supporting evidence. The hypotheses in Table 1 demonstrate the types of scientific insights that the multi-agent artificial intelligence system may generate from mass spectrometry data.

In one example hypothesis generated by Gemini 2.0 Flash, the presence of 1H-Phenalen-1-one or 9H-Fluoren-9-one (ID 44, MW 180) exclusively in Orgueil and LEW 85311, and the presence of Biphenyl (ID 43, MW 154) also in the same meteorites, suggests a unique chemical environment or alteration history shared by these samples, potentially indicating a similar formation region within the early solar system. In another example hypothesis generated by Claude 3.5 Sonnet, the exclusive detection of 1,2,3,4-tetrahydro phenanthrene (ID 36) in Orgueil and Jbilet Winselwan, along with phenanthrene/anthracene (ID 42), suggests a specific hydrogenation pathway in certain meteorite parent bodies that resulted in partial reduction of aromatic systems. In a further example hypothesis generated by Claude 3.5 Sonnet, the detection of possible terpenes exclusively in soil samples indicates that complex branched isoprenoid structures require enzymatic biosynthesis and are not readily formed through abiotic processes in space, making them reliable biomarkers.

TABLE 1
Selected High-Scoring Hypotheses Generated by
the Multi-Agent Artificial Intelligence System

| Hypothesis | Statement | Key Datapoints |
|---|---|---|
| Gemini 2.0 | The presence of 1H-Phenalen-1-one or 9H-Fluoren-9- | 1H-Phenalen-1-one or |
| Flash | one (ID 44, MW 180) exclusively in Orgueil and LEW | 9H-Fluoren-9-one (ID |
| | 85311, and the presence of Biphenyl (ID 43, MW 154) | 44, MW 180): Orgueil, |
| | also in the same meteorites, suggests a unique | LEW 85311; Biphenyl |
| | chemical environment or alteration history shared by | (ID 43, MW 154): |
| | these samples, potentially indicating a similar | Orgueil, LEW 85311 |
| | formation region within the early solar system. | |
| Claude 3.5 | The exclusive detection of 1,2,3,4-tetrahydro | ID 36 (1,2,3,4-tetrahydro |
| Sonnet | phenanthrene (ID 36) in Orgueil and Jbilet Winselwan, | phenanthrene) in Orgueil |
| | along with phenanthrene/anthracene (ID 42), suggests | and Jbilet Winselwan; ID |
| | a specific hydrogenation pathway in certain meteorite | 42 |
| | parent bodies that resulted in partial reduction of | (phenanthrene/anthracene) |
| | aromatic systems. | in Orgueil, LEW 85311 |
| Claude 3.5 | The detection of possible terpenes exclusively in soil | IDs 4, 17, and 18 |
| Sonnet | samples indicates that complex branched isoprenoid | (possible terpenes) were |
| | structures require enzymatic biosynthesis and are not | found only in soil |
| | readily formed through abiotic processes in space, | samples (Iceland Soil, |
| | making them reliable biomarkers. | Atacama, Utah soil, |
| | | GSFC soil) |

Table 2 presents the average scores and their corresponding standard deviation assigned by astrobiology experts to hypotheses generated by two large language models: Claude Sonnet 3.5 and Gemini 2.0 Flash. Over 10 iterations of the multi-agent artificial intelligence system, Claude Sonnet 3.5 produced 48 hypotheses while Gemini 2.0 Flash produced 101 hypotheses. Each hypothesis was evaluated on a 0 to 10 scale across six distinct criteria.

TABLE 2
Human Expert Evaluation Scores for Hypotheses Generated
by Claude Sonnet 3.5 and Gemini 2.0 Flash

| Criteria | Claude Sonnet 3.5 | Gemini 2.0 Flash |
|---|---|---|
| Novelty | 2.75 ± 0.75 | 4.26 ± 1.87 |
| Consistency with the literature | 7.60 ± 1.91 | 6.19 ± 2.88 |
| Clarity and precision | 7.20 ± 2.30 | 5.92 ± 2.86 |
| Empirical Support | 6.75 ± 2.63 | 5.79 ± 2.86 |
| Scope & Generalizability | 7.60 ± 1.91 | 6.01 ± 2.80 |
| Predictive Power | 7.60 ± 1.91 | 5.86 ± 2.68 |
| Overall Average | 6.58 ± 1.74 | 5.67 ± 0.64 |

As shown in Table 2, the multi-agent artificial intelligence system powered by Claude Sonnet 3.5 achieved an average score of 6.58±1.74, while Gemini 2.0 Flash achieved an average score of 5.67±0.64. Claude Sonnet 3.5 demonstrated higher scores for consistency with the literature, clarity and precision, empirical support, scope and generalizability, and predictive power. Gemini 2.0 Flash demonstrated a higher average novelty score compared to Claude Sonnet 3.5.

Among the 101 hypotheses generated by Gemini 2.0 Flash, 36 were determined to be plausible by the expert, and of these, 24 were flagged as novel. Among the 48 hypotheses generated by Claude Sonnet 3.5, 24 were determined to be plausible by the expert, with none flagged as novel. The results indicate that a large language model with stronger agent collaboration capabilities may produce hypotheses that are more consistent and exhibit fewer logical errors, while a large language model with a larger context window may facilitate more creative connections across broader knowledge bases by enabling access to more extensive background information.

The multi-agent artificial intelligence system enables hypothesis generation without requiring prior domain expertise in mass spectrometry data interpretation. The system incorporates scientific literature as background context for the agents, enabling the agents to interpret the mass spectrometry data within the framework of existing research. The literature-integrated reasoning performed by the agents enables the generation of insights that are informed by prior scientific findings. The system makes advanced analysis accessible to users who may not have specialized training in mass spectrometry or astrobiology by automating the pattern identification, hypothesis generation, and literature review processes.

The multi-agent artificial intelligence system operates on both mass spectrometry data and text data for comprehensive analysis. The system receives mass spectrometry data in a structured format and research papers that are converted to a text format. The agents process both data types to generate hypotheses that are supported by the mass spectrometry data and consistent with the scientific literature. The multimodal data processing capability enables the system to integrate quantitative information from the mass spectrometry data with qualitative information from the research papers.

The multi-agent artificial intelligence system may be applied to datasets beyond those specifically described herein. The system architecture is compatible with mass spectrometry data from various instruments and may be adapted for analysis of different sample types and compound classes. The system may be applied to analyze mass spectrometry data from sample return missions, laboratory experiments, and other sources where hypothesis generation from complex molecular data is desired. The broad applicability of the system enables researchers in various fields to utilize the multi-agent approach for hypothesis generation from mass spectrometry data.

A number of implementations have been described. Nevertheless, it will be understood that various modifications may be made without departing from the spirit and scope of the disclosure. Accordingly, other implementations are within the scope of the following claims.

1. A method for generating hypotheses from mass spectrometry data using a multi-agent artificial intelligence system, comprising:
receiving, via a user interface, mass spectrometry data, one or more research papers, and instructions from a user;
analyzing, via a data analyst agent, the mass spectrometry data to identify patterns, unexpected findings, and potential contamination;
delegating, via a planner agent, segments of the mass spectrometry data to a plurality of scientist agents based on the analysis from the data analyst agent;
generating, via the plurality of scientist agents, hypotheses based on the delegated segments of the mass spectrometry data;
consolidating, via an accumulator agent, the hypotheses generated by the plurality of scientist agents by discarding duplicate hypotheses;
reviewing, via a literature review agent, literature relevant to each of the consolidated hypotheses;
evaluating, via a critic agent, the consolidated hypotheses based on the reviewed literature; and
providing feedback from the critic agent to the data analyst agent for iterative refinement of subsequent analyses.

2. The method of claim 1, wherein analyzing the mass spectrometry data comprises identifying polycyclic aromatic hydrocarbon distribution patterns in the mass spectrometry data.

3. The method of claim 2, wherein the polycyclic aromatic hydrocarbon distribution patterns include methylated polycyclic aromatic hydrocarbons and unsubstituted polycyclic aromatic hydrocarbons.

4. The method of claim 1, wherein the plurality of scientist agents comprises three scientist agents, each scientist agent assigned to analyze a distinct research area within the mass spectrometry data.

5. The method of claim 4, wherein the distinct research areas include:
polycyclic aromatic hydrocarbon distribution patterns;
sulfur-containing and oxygen-containing compounds; and
alkylated aromatic compounds.

6. The method of claim 1, wherein generating the hypotheses comprises outputting the hypotheses in a structured format including a hypothesis statement and key data points supporting the hypothesis.

7. The method of claim 6, wherein the structured format comprises a JSON object containing an identifier, the hypothesis statement, and the key data points.

8. The method of claim 1, wherein reviewing literature relevant to each of the consolidated hypotheses comprises searching a scientific literature database for papers corresponding to each hypothesis and synthesizing key insights from search results.

9. The method of claim 1, wherein evaluating the consolidated hypotheses comprises:
assessing alignment of each hypothesis with the mass spectrometry data;
evaluating scientific rigor and theoretical basis of each hypothesis; and
assessing novelty and specificity of each hypothesis.

10. The method of claim 1, wherein the one or more research papers are converted to a text format and provided as background context to the data analyst agent and the plurality of scientist agents.

11. The method of claim 1, wherein the mass spectrometry data comprises data obtained from two-dimensional gas chromatography coupled with high-resolution mass spectrometry.

12. A system for generating hypotheses from mass spectrometry data, comprising:
a processor;
a memory coupled to the processor; and
a storage coupled to the processor, the storage storing instructions that, when executed by the processor, cause the processor to:
receive mass spectrometry data, one or more research papers, and instructions via a user interface;
execute a data analyst agent to analyze the mass spectrometry data and identify patterns and unexpected findings;
execute a planner agent to delegate segments of the mass spectrometry data to a plurality of scientist agents;
execute the plurality of scientist agents to generate hypotheses based on assigned segments of the mass spectrometry data;
execute an accumulator agent to consolidate the hypotheses by removing duplicate hypotheses;
execute a literature review agent to search for and summarize literature relevant to each consolidated hypothesis; and
execute a critic agent to evaluate the consolidated hypotheses and provide feedback to the data analyst agent for iterative refinement.

13. The system of claim 12, wherein the instructions further cause the processor to convert the one or more research papers to a text format and provide the text format as background context to the data analyst agent and the plurality of scientist agents.

14. The system of claim 12, wherein the plurality of scientist agents comprises three scientist agents, each scientist agent configured to analyze a distinct research area within the mass spectrometry data.

15. The system of claim 14, wherein the distinct research areas comprise:
polycyclic aromatic hydrocarbon distribution patterns;
sulfur-containing and oxygen-containing compounds; and
alkylated aromatic compounds.

16. The system of claim 12, wherein the instructions cause the processor to execute the critic agent to evaluate the consolidated hypotheses by:
assessing alignment of each hypothesis with the mass spectrometry data;
evaluating scientific rigor and theoretical basis of each hypothesis;
assessing novelty and specificity of each hypothesis; and
generating structured feedback for iterative refinement.

17. The system of claim 12, wherein the instructions cause the processor to execute the literature review agent to search a scientific literature database for papers corresponding to each consolidated hypothesis and synthesize key insights from search results.

18. A non-transitory computer-readable medium storing instructions that, when executed by a processor, cause the processor to perform operations comprising:
receiving mass spectrometry data and research papers;
analyzing, via a data analyst agent, the mass spectrometry data to identify patterns;
delegating, via a planner agent, data segments to a plurality of scientist agents;
generating, via the plurality of scientist agents, hypotheses based on the delegated data segments;
consolidating, via an accumulator agent, the generated hypotheses;
reviewing, via a literature review agent, literature for each consolidated hypothesis;
evaluating, via a critic agent, the consolidated hypotheses; and
iteratively refining the analysis based on feedback from the critic agent.

19. The non-transitory computer-readable medium of claim 18, wherein the operations further comprise converting the research papers to a text format and providing the text format as background context to the data analyst agent and the plurality of scientist agents.

20. The non-transitory computer-readable medium of claim 19, wherein the plurality of scientist agents comprises three scientist agents, each scientist agent configured to analyze a distinct research area within the mass spectrometry data.
